(19) United States
(12) Patent Application Publication
(10) Pub. No.: US 2003/0031715 A1
(43) Pub. Date: Feb. 13, 2003

(54) Pharmaceutical applications of hydrotropic agents, polymers thereof, and hydrogels thereof

(76) Inventors: Kinam Park, West Lafayette, IN (US); Ghanashyam Acharya, Saitama (JP); Jaehwi Lee, West Lafayette, IN (US); Sang Cheon Lee, West Lafayette, IN (US)

Correspondence Address: MEDICUS ASSOCIATES AND JAMES H MEADOWS, 5355 MIRA SORRENTO PL., SUITE 100, SAN DIEGO, CA, 92121

(21) Appl. No.: 09/975,800

(22) Filed: Oct. 11, 2001

Related U.S. Application Data
(60) Provisional application No. 60/239,455, filed on Oct. 11, 2000.
(60) Provisional application No. 60/294,957, filed on May 31, 2001.

(51) Int. Cl. A61K 31/34; A61K 31/56; A61K 31/337; A61K 31/137; A61K 9/14

(52) U.S. Cl. 424/486; 514/171; 514/462; 514/449; 514/651

(57) ABSTRACT

The present invention is directed to compounds effective for increasing the water solubility of poorly soluble drugs. Hydrotropic agents are identified, such as for increasing the solubility of paclitaxel. Polymerizable monomers of the hydrotropic agents are prepared and hydrotropic polymers formed from such monomers are generated. Both the monomers and resulting polymers increase the solubility of poorly soluble drugs. In some cases, the hydrotropic polymers are more effective at increasing solubility at low concentrations relative to a corresponding amount of the hydrotropic agent precursor. Additionally, the hydrotropic polymers (hytrops) can be crosslinked to yield hydrotropic hydrogels (hytrogels) capable of solubilizing a drug. The hytrogels can further be employed to generate micro- and nano-particle suspensions of a poorly soluble drug. The water solubility of paclitaxel can be increased by four orders of magnitude using compounds of the invention. Large molecular weight compounds, such as the hytrops and hytrogels, are expected to have low levels of absorption in the gastrointestinal tract, thereby making them particularly preferred for oral delivery of poorly soluble drugs.

### REFERENCE TO RELATED APPLICATIONS

The present application is related to U.S. provisional application 60/ 239,455, filed Oct. 11, 2000, and U.S. provisional application 60/294,957, filed May 31, 2001.

### FIELD OF THE INVENTION

The present invention relates to chemical compositions and methods of drug delivery, particularly those relating to delivery of poorly soluble drugs.

### BACKGROUND OF THE INVENTION

Many drugs and drug candidates are poorly water-soluble, which limits their clinical applications. Increasing numbers of newly developed drugs are poorly water-soluble and such poor water-solubility causes significant problems in producing formulations of a sufficiently high bioavailability with reproducible effects. (Müller, R. H. et al. 1998; Löbenberg, R. et al. 2000) A “poorly water-soluble” drug (or simply “poorly soluble” drug) refers to a “practically insoluble” drug in the U.S. Pharmacopeia., and is defined as a drug having a water solubility of less than 0.1 mg/ml (or 100 μg/ml). Whenever the drug concentration is much less than 0.1 mg/ml, its oral absorption is usually poor or at least inconsistent. (Macheras, P. et al. 1995)

The water-solubility of a drug depends on its hydrophilicity-lipophilicity balance, which is often measured by partition of the drug between two immiscible solvents -octanol and water. The partition coefficient (or distribution coefficient) is defined as:

Partition Coefficient=log (C_{O}/C_{W}) where C_{O} and C_{W} are the equilibrium concentrations of the drug in octanol and water, respectively. Thus, a drug with a partition coefficient of 2 means that it dissolves in octanol 100 times more than in water. The concept of partition coefficient is important because the absorption of drugs from the gastrointestinal tract is linearly related to partition coefficient rather than to water solubility. This is due to the fact that drugs have to pass through the lipid cell bilayers for absorption, and the lipophilicity of cell bilayers can be approximated by octanol. As shown in Table 1, water solubilities and partition coefficients do not have a linear relationship, even though, in general, drugs having lower water solubility have a higher partition coefficient. Caution should be exercised in applying this general rule, because if a drug is too hydrophobic with a very high partition coefficient, it is too poorly water-soluble, thereby limiting absorption. Therefore, in terms of drug absorption and subsequent bioavailability, a higher partition coefficient is not necessarily better. If the water solubility of drugs having a high partition coefficient can be increased, the bioavailability of the drug is also expected to increase since absorption is linearly dependent on the total amount of a dissolved drug.

TABLE 1
Representative drugs having poor
water-solubility (i.e., water-solubility
of less than 100 μg/ml at 37° C.)

| | M.W. | Water Solubility | | Partition |
|---|---|---|---|---|
| Drug | (g/mol) | (μM) | (μg/ml) | Coefficient |
| Tolbutamide | 270.3 | 202.6 | 54.8 | 0.40 |
| Thalidomide | 258.2 | 77.5 | 20.0 | 0.64 |
| Chloramphenicol | 323.1 | 199.0 | 64.0 | 1.08 |
| Diclofenac | 296.1 | 10.1 | 3.0 | 1.12 |
| Digoxin | 780.9 | 38.4 | 30.0 | 1.26 |
| Hydrocortisone | 362.5 | 202.9 | 73.6 | 1.52 |
| Phenacetin | 179.2 | 202.8 | 36.3 | 1.55 |
| Dexamethasone | 392.5 | 25.5 | 10.0 | 1.95 |
| Quinidine | 324.4 | 198.1 | 64.3 | 1.99 |
| Griseofulvin | 352.8 | 19.8 | 7.0 | 2.07 |
| Nifedifine | 346.3 | 28.9 | 10.0 | 2.20 |
| Phenytoin | 252.3 | 79.3 | 20.0 | 2.47 |
| Spironolactone | 416.6 | 72.0 | 30.0 | 2.78 |
| Mebendazole | 295.3 | 1.7 | 0.5 | 2.83 |
| Chlorpromazine | 318.9 | 94.1 | 30.0 | 3.17 |
| Nicardipine | 479.5 | 7.1 | 3.4 | 3.62 |
| Norethindrone | 298.4 | 32.9 | 9.8 | 3.15 |
| Paclitaxel | 853.9 | 0.4 | 0.3 | 3.62 |
| Estrone | 270.4 | 7.4 | 2.0 | 3.69 |
| Reserpine | 608.7 | 1.6 | 1.0 | 3.73 |
| Progesterone | 314.5 | 3.8 | 1.2 | 3.84 |
| Terfenadine | 471.7 | 152.8 | 72.1 | 4.05 |
| Trifluoperazine | 407.5 | 44.7 | 18.2 | 4.15 |
| Indomethacin | 357.8 | 55.9 | 20.0 | 4.27 |
| Pimozide | 461.5 | 2.2 | 1.0 | 4.50 |
| Cinnarizine | 368.5 | <1.0 | <0.4 | 4.50 |
| Diethylstilbestrol | 268.4 | 7.5 | 2.0 | 4.50 |
| Flunarizine | 404.5 | 1.0 | 0.4 | 4.70 |
| Tamoxifen | 371.5 | 1.1 | 0.4 | 4.90 |
| Itraconazole | 705.6 | 2.8 | 2.0 | 5.66 |
| Rapamycin | 914.2 | 3.3 | 3.0 | — |

Other poorly soluble drugs not listed in Table 1 include alprostadil, amphotericin B, camptothecin, cosalane, chloramphenicol, cyclosporine, dexamethasone, diazepam, digoxin, epirubicin, glucocorticosteroids, HIV-1 protease inhibitors, palmitoylrhizoxin, p-boronophenylalanine, pregnanolone, and propofol.

To illustrate the importance of water-solubility, paclitaxel (underlined in Table 1) is taken as an example. Paclitaxel has an exceedingly low water solubility and a high partition coefficient. Optimally effective use of paclitaxel (brand name TAXOL) in cancer therapy has been hindered by its low water-solubility. This low solubility requires special formulation utilizing ethanol and Cremophore EL (polyoxyethylated castor oil), which has toxic side effects, such as lethal anaphylaxis. This has made it difficult to evaluate paclitaxel in preclinical tumor model systems. (Leung, S. Y. et al. 2000) In addition, the cosolvent mixture is diluted before intravenous (i.v.) administration in isotonic saline solution and remains stable for only three hours. (Floyd, A. G. et al. 1998)

The poor bioavailability of poorly water-soluble drugs becomes even worse when the drug is given orally. (Mani, S. et al. 1998) Since oral administration is the most convenient method of delivering drugs and is used for the majority of drugs, developing a method for increasing the water-solubility of poorly soluble drugs is highly important. Increasing the water-solubility of poorly water-soluble drugs should allow development of effective oral dosage forms. Dissolution of the active ingredient from a conventional dosage form (e.g., tablet or suspension) is one of the most critical steps in drug absorption leading to bioavailability. For poorly water-soluble drugs, dissolution in aqueous media is often the primary limitation. When the aqueous solubility of a drug is smaller than 0.1 mg/ml, dissolution of the drug is too slow for effective absorption of the drug. (Macheras, P. et al. 1995) Moreover, systemic delivery of paclitaxel in large doses is limited by hematologic toxicity, neutropenia, and dose-dependent neurotoxicity. The ability to deliver a smaller amount of paclitaxel by oral administration may reduce the toxicity associated with large doses given i.v. every few weeks, since oral administration generally enjoys better compliance. An increase in the water-solubility of poorly soluble drugs should provide new avenues of drug delivery that have not been possible before.

Current approaches for improving the water-solubility of poorly soluble drugs are listed below:

Synthesis of prodrugs and analogs

Physical modification of drugs

Use of cosolvents

Emulsions, micelles, and liposomes

Complexation approach

Solid dispersion technology

Use of hydrotropic agents (hydrotropes)

Synthesis of Prodrugs and Analogs

The prodrug approach is highly viable, and a number of prodrugs have been studied. For example, paclitaxel prodrugs having higher water solubility have been synthesized. (Nicolaou, K. C. et al. 1993; Pendri, A. et al. 1998) Such paclitaxel analogs having increased water-solubility, however, showed diminished anticancer activity upon oral administration. The main limitation of the prodrug or analog approach is that the prodrugs and analogs are regarded as “new chemical entities”, which limits their attractiveness due to the associated prolonged clinical and regulatory delays.

Physical Modification of Drugs

The aqueous solubility of hydrophobic drug particles increases as the particle size decreases. The Kelvin equation, which was developed to describe the increase in vapor pressure across a curved surface of small liquid drops, has been applied to describe the solubility of drug particles:

1n(Cᵣ/C_{∞})=(2Mγₛₗ)/(RTρr)

where Cᵣ and C_{∞} are the respective solubilities of drug particles having radius r and infinitely large radius (which is the case for any particles over a few microns in size), M is the molecular weight, γₛₗ is the solid-liquid surface tension, R is the gas constant, T is the temperature, and ρ is the density of the solid. The measured solubilities with different particle sizes are metastable equilibrium states, which eventually return to the stable state, i.e., the true equilibrium solubility. The equation implies that large particles (or crystals) will grow at the expense of smaller ones, which is known as Ostwald ripening.

Microparticulate preparations of poorly soluble drugs are commonly prepared by spray drying, emulsion-solvent extraction, microfluidization, high pressure homogenization, ball milling, media milling, jet milling, and rapid expansion from supercritical fluid. Paclitaxel particles less than 1 μm have been prepared and are called “nanosuspensions”. (Müller, R. H. et al 1998) The primary limitation of this approach is that the increase in water-solubility is less than an order of magnitude in most cases.

Use of Cosolvents

Cosolvent systems can increase the water-solubility of a drug significantly, but the choices of biocompatible solvents are limited, such as to glycerin, propylene glycol, poly(ethylene glycol)s, dimethylsulfoxide, N,N-dimethylformamide, cremophore, and ethanol. Cosolvent systems are not as biocompatible as aqueous solutions.

Emulsions, Micelles, and Liposomes

Emulsions are dispersions of droplets of one liquid in another immiscible liquid. Emulsifiers are, in general, surfactants, and are employed to prevent the droplets from coalescing. For delivery of poorly soluble drugs, oil-in-water (o/w) emulsions are usually used. Commonly used oil cores are triolein, triglyceride, propyleneglycol dicaprylate, and soybean oil.

Liposomes and micelles also have been studied quite extensively for delivery of important poorly soluble drugs, such as paclitaxel (Alkan-Onyuksel, H. et al. 1994; Sharma, A. et aL 1994). The main limitation of this approach is that the liposomes and micelles tend to have poor stability. The liposomes are typically vesicles composed of naturally occurring or synthetic phospholipids. The vesicles are spherical or ellipsoidal closed bilayer structures. The bilayer structure can be single- or multi-compartment. The size can also vary from smaller than 1 μm to larger than 10 μm. The typical diameters of small unilamellar, large unilamellar, and multilamellar liposomes are 0.1 μm, 1 μm and 5 μm, respectively. Micelles are aggregates of detergent molecules in aqueous solution. Detergents are water-soluble, surface-active agents composed of a hydrophilic head group and a hydrophobic or lipophilic tail group. They can also align at aqueous/nonaqueous interfaces, reducing surface tension, increasing miscibility, and stabilizing emulsions.

Complexation

The complexation approach has been frequently used to increase the water solubility of poorly soluble drugs. The most common complexing ligands are cyclodextrins, caffeine, urea, poly(ethylene glycol)s, N-methylglucamide. Cyclodextrins are unique since they increase the water-solubility of poorly soluble drugs by fitting them into the hydrophobic cavity of the cyclodextrin molecule. The drugs tend to precipitate out upon dilution of the cyclodextrins.

Solid Dispersion Technology

Solid dispersion is the dispersion of a poorly soluble drug in an inert polymeric carrier (such as PVP) at solid state prepared by the melting or solvent method. This method requires melting of the drug or the use of organic solvents (Chiou, W. L. et al. 1971; Ford, J. L. 1986; Serajuddin, A. T. M. 1999; Habib, M. J. et al 2001).

Use of Hydrotropic Agents (Hydrotropes)

Hydrotropy refers to a solubilization process whereby the addition of large amounts of a second solute results in an increase in the aqueous solubility of a poorly soluble compound (Coffman, R. E. et al. 1996). Hydrotropic agents (or hydrotropes) are compounds that, at high concentrations, solubilize poorly water-soluble molecules in water (Saleh, A. M. et al. 1986). At concentrations higher than the minimal hydrotrope concentration, hydrotropic agents self-associate and form noncovalent assemblies of lowered polarity, i.e., nonpolar microdomains, which solubilize hydrophobic solutes (Dhara, D. et al. 1999). The self-aggregation of hydrotropic agents is different from surfactant self-assemblies (i.e., micelles) in that hydrotropes form planar or open-layer structures instead of compact spheroid assemblies (Srinivas, V. et al. 1998). Hydrotropic agents are structurally characterized by having a short, bulky, compact moiety (such as an aromatic ring), while surfactants have long hydrocarbon chains. In general, hydrotropic agents have a shorter hydrophobic segment, leading to higher water solubility, than do surfactants. The hydrotropy is suggested to be superior to other solubilization methods, such as micellar solubilization, miscibility, cosolvency, and salting-in, because the solvent character is independent of pH, has high selectivity, and does not require emulsification (Kumar, M. D. et al. 2000).

Examples of hydrotropic materials used as excipients in the literature are sodium salicylate, sodium gentisate, sodium glycinate, sodium benzoate, sodium toluate, sodium ibuprofen, pheniramine, lysine, tryptophan, and isoniazid (see Saleh, A. M. et al. 1986). Each hydrotropic agent is effective in increasing the water solubility of selected hydrophobic drugs; no universal hydrotropic agent has been found effective to solubilize all hydrophobic drugs. Thus, finding the right hydrotropic agents for a poorly soluble drug requires screening a large number of candidate hydrotropes. However, once the effective hydrotropic agents are identified for a series of structurally different drugs, the structure-activity relationship can be established.

Of the various approaches discussed above, the hydrotrope approach is a highly promising new method with great potential for poorly soluble drugs in general. For instance, should the solubility of paclitaxel be increased by 2-4 orders of magnitude in the presence of hydrotropic compounds, the oral absorption and subsequent bioavailability is also expected to increase by a similar extent. The increase in solubility is also expected to be beneficial in overcoming the adverse effects of P-glycoproteins in the GI tract, due to excess drug saturating the P-glycoproteins. This consideration is especially important for those conditions that are largely untreatable due to multi-drug resistance, e.g., certain breast cancers.

Using hydrotropic agents is one of the easiest ways of increasing water-solubility of poorly soluble drugs, since it only requires mixing the drugs with the hydrotrope in water. The hydrotrope approach does not require chemical modification of hydrophobic drugs, use of organic solvents, or preparation of emulsion systems. Despite these advantages, hydrotropes have not been widely explored for increasing the water solubility of poorly soluble drugs. The main reason for this may be a concern that the use of low molecular weight hydrotropic agents may result in the co-absorption of a significant amount of the hydrotropic agent either from the GI tract after oral administration or from the bloodstream after parenteral injection.

Previously, the synthesis of polymers based on polymerizable derivatives of 5-oxo-pyrrolidinecarboxylic acid and pyrrolidonyl oxazoline monomers has been reported. (U.S. Pat. Nos. 4,933,463; 4,981,974 and 5,008,367 to Dandreaux et al.; U.S. Pat. Nos. 4,946,967 and 4,987,210 to Login et al.) The structures of the aforementioned polymers are modifications of polyvinylpyrrolidone (PVP), a well-known synthetic polymer having a variety of applications. Steric crowding between the hydrophilic pyrrolidone ring and hydrophobic hydrocarbon backbone of the PVP polymer was proposed to limit complexation of the polymer with other molecules, especially when dipole-dipole interactions are involved. (Dandreaux et al.). Accordingly, the investigators synthesized pyrrolidone-containing polymers wherein the pyrrolidone ring is spaced away from the polymer backbone. The resulting polymers reportedly show an increase in water solubility of selected organic compounds. Since the structures of these polymers are based on PVP, the range of compounds is very limited. Moreover, the aforementioned PVP-based polymers are not believed to be particularly water-soluble and, therefore, are not expected to display pronounced hydrotropic properties.

Another class of compounds, e.g., represented by PEGs and water-soluble carbohydrates, reportedly has been studied for the ability to increase water solubility of certain structurally similar drugs, particularly quinazoline-, nitrothiazole-, and indolinone-based compounds. (U.S. Pat. No. 6,248,771 to Shenoy et al.) The combination of a pharmacologically active compound, such as cyclosporin, with a monoester made from a fatty acid and a polyol, such as a saccharide, also has been proposed. (U.S. Pat. No. 5,756,450 to Hahn et al.) The use of peptides, such as gelatins, in formulations to increase the solubility of the drug has been suggested. (U.S. Pat. No. 5,902,606 to Wunderlich et al.)

A need exists for new classes of hydrotropic compounds having the desired properties of increasing the water solubility of poorly soluble drugs. It is especially desired to identify hydrotropic compounds having high molecular weights so that they are not co-absorbed with the poorly soluble drug.

### SUMMARY OF THE INVENTION

The present invention is for novel compositions of matter and methods employing hydrotropic compounds to increase the aqueous solubility of poorly soluble drugs. Thus, a pharmaceutical composition of the invention comprises a pharmacologically effective amount of a poorly soluble drug and a solubilizing compound. The solubilizing compound is selected from among hydrotropic agent monomers, hydrotropic polymers, and hydrotropic hydrogels, and further includes at least one hydrophobic moiety.

In a preferred aspect, novel higher molecular weight hydrotropic polymers, copolymers, and gels, obtained as the linear, branched, and crosslinked molecules, are employed as the solubilizing compound. Specifically, the present invention enables the identification of a hydrotropic polymer (trademark HYTROP) and a hydrotropic hydrogel (trademark HYTROGEL), i.e., a crosslinked hydrotropic polymer, suitable for formulation with and/or co-administration with a given drug. The structure of the hydrotropic compound (polymer, copolymer or hydrogel) is based on the structures of known hydrotropic agents effective in solubilizing the drug. The invention is illustrated particularly using paclitaxel, which is a model poorly soluble drug.

A solubilizing compound of the present invention contains a hydrophobic moiety, which is capable of breaking up water structure and/or interacting in an energetically favorable manner with a hydrophobic drug. The hydrophobic moiety is preferably selected from among substituted and unsubstituted aryl groups, substituted and unsubstituted nitrogen heterocycles, alkyl groups, alkylene groups, aralkyl groups, and methacryloyl groups. More preferably, the hydrophobic moiety is a substituted or unsubstituted pyridyl group, e.g., a nicotinamide derivative. Most preferably, the hydrophobic moiety is selected from N,N-diethylnicotinamide, N-picolylnicotinamide, N-allylnicotinamide, sodium salicylate, 2-methacryloyloxyethyl phosphorylcholine, resorcinol, N,N-dimethylnicotinamide, N-methylnicotinamide, butylurea, pyrogallol, 3-picolylacetamide, procaine HCl, nicotinamide, pyridine, 3-picolylamine, sodium ibuprofen, sodium xylenesulfonate, and ethyl carbamate.

A hydrotropic polymer or copolymer of the invention has a block, graft, alternating or random arrangement of monomer units. It typically has an acrylate or methacrylate backbone, and may or may not contain a spacer group in order to separate the hydrophobic moiety from the polymer backbone. Exemplary hydrotropic agent monomer units used to form the polymer or copolymer are polymerizable derivatives of nicotinamide, N-substituted nicotinamide, pyridinium, N-substituted pyridinium, benzyl, urea, thiourea, pyridone, pyrimidone, melamine, pyridine, pyrazine, nicotine, triazine, salicylamide, salicylic acid, and sulfimide. More particularly, at least one hydrotropic agent monomer unit is a vinyl derivative of ibuprofen, nicotinamide, salicylic acid, N-picolylnicotinamide, salicylaldehyde, N,N′-dimethylnicotinamide, N,N′-diethylnicotinamide, or pyridine.

A hydrotropic hydrogel of the invention is capable of increasing water solubility of a poorly soluble drug. The hydrogel is formed by polymerizing at least one hydrotropic agent monomer in the presence of a crosslinking agent and typically exhibits solubilizing power comparable to a corresponding polymer. Suitable hydrophobic moieties of the hydrogel are as described above.

A method of increasing water solubility of a hydrophobic compound, generally, comprises combining the hydrophobic compound with a solubilizing compound from among hydrotropic agents, hydrotropic agent monomers, hydrotropic polymers, and hydrotropic hydrogels, wherein the solubilizing compound has a hydrophobic moiety.

Also contemplated is a method of administering a poorly soluble drug to a patient in need thereof. The method comprises administering to the patient a composition containing the drug and a solubilizing compound as excipient. The excipient can be a hydrotropic agent, hydrotropic agent monomer, hydrotropic polymer and/or hydrotropic hydrogel. The solubilizing compound includes a hydrophobic moiety that assists in increasing the solubility of the drug. Preferably, administration is by the oral route, although other routes are contemplated. Formulations employing hydrotropic polymers or hydrogels are particularly preferred.

Since the exact mechanisms involved in increasing the water-solubility of poorly soluble drugs with hydrotropic agents are not known, it is often difficult to predict the structural requirements of hydrotropes suitable for solubilizing a given drug. Thus, the most rational approach to the synthesis of hydrotropic polymers involves utilizing the most promising low molecular weight hydrotropic agents as monomers. As described more fully hereinafter, more than 50 hydrotropic agents for paclitaxel have been screened to identify several effective hydrotropic agents. Based on the structures of the identified hydrotropic agents, several hydrotropic polymers and hydrotropic hydrogels for paclitaxel have been synthesized. The hydrotropic polymers were observed to increase paclitaxel solubility by 3 orders of magnitude or more. Of course, the same approach can be used for the synthesis of hydrotropic polymers and hydrogels suitable for other poorly soluble drugs. The availability of new hydrotropic polymers and hydrogels should permit development of novel delivery systems for many drugs and drug candidates where applications have been limited previously due to their poor water solubilities.

### BRIEF DESCRIPTION OF THE DRAWINGS

FIG. 1 depicts paclitaxel solubility (mg/ml) as a function of the molar concentration of N,N-diethylnicotinamide. The uppermost paclitaxel solubility (512.6 mg/ml) reached at 5.95 M of N,N-diethylnicotinamide corresponds to 0.60 M. Paclitaxel M.W.=853.9 g/mol.

FIG. 2 shows a comparison of the hydrotropic properties for 6-(4-vinylbenzyloxy)-N-picolylnicotinamide (monomer) and its polymer at different monomer concentrations as applied to increasing the water solubility of paclitaxel.

FIG. 3 depicts release of paclitaxel from a hydrotropic polymer formulation. The concentration of dissolved paclitaxel is high in the diffusion layer. Dissolved paclitaxel molecules diffuse (A) through the aqueous layer. Paclitaxel molecules may precipitate (B) to form fine particles, which rapidly redissolve (C) due to their fine particle sizes. Dissolved paclitaxel molecules are absorbed through the cell membrane (D).

### DETAILED DESCRIPTION OF THE INVENTION

The present invention affords convenient compounds and methods for increasing the solubility of a poorly soluble pharmacologically active compound, i.e., a drug. As used herein, a “poorly soluble” drug has a water solubility of less than about 100 μg/ml at 37° C. Representative drugs are paclitaxel, griseofulvin, progesterone, and tamoxifen. Other compounds are listed in Table 1. The terms “pharmacologically active”, “pharmaceutically acceptable”, or “pharmaceutical”, as used herein, refer to solutions or components that do not prevent the pertinent compound from exerting a beneficial therapeutic effect. Examples of such compounds are too abundant to enumerate and are available in a variety of sources, e.g., Merck Index, U.S. Pharmacopeia, etc., which are incorporated herein by reference. Any side effects associated with a drug vary with the drug and for different diseases and conditions.

The present invention employs a solubilizing compound to increase the inherent aqueous solubility of a target drug. The solubilizing compound is selected from among hydrotropic agent monomers, hydrotropic polymers, and hydrotropic hydrogels, which include at least one hydrophobic moiety.

As used herein, the term “hydrotropic agent” refers to a material that increases the affinity of another substance, such as a pharmaceutical compound, for water. The resulting concentration of the substance in water is effectively greater in the presence of hydrotropic agent than in its absence. Likewise, the observable solubility of the substance in water increases in the presence of hydrotropic agent.

As used herein, the term “hydrotropic agent monomer”, “hydrotropic monomer”, and the like, refers to a polymerizable form of a hydrotropic agent, which itself may or may not be polymerizable. The term “hydrotropic polymer” and “hydrotropic copolymer”, and the like, refers to a polymeric product that has been polymerized from one or more hydrotropic monomer(s), such as one bearing a polymerizable vinyl group.

As used herein, a “hydrotropic hydrogel” is a crosslinked hydrotropic polymer or copolymer, which is capable of increasing the solubility of a poorly soluble drug.

I. Hydrotropic Agents

A. Low Molecular Weight Hydrotropic Agents for Paclitaxel

Due to its noted therapeutic potential and very low water solubility, paclitaxel (PTX) is a prime candidate for study as a model drug compound for testing with the present invention. Accordingly, a large number of hydrotropic agent candidates have been examined for their ability to increase the water solubility of paclitaxel. Table 2 lists the agents tested and the corresponding water solubilities of paclitaxel determined in the presence of those agents. The minimum hydrotrope concentration (MHC) required to solubilize a compound is different for different hydrotropes, but a preliminary study suggests that even good hydrotropes have an MHC of approximately 3 M. For this reason, in the comparison of hydrotropic properties for various agents, 3.5 M was chosen for study. The concentrations of some agents in Table 2 are less than 3.5 M, which is simply due to the limited solubility of those agents.

The hydrotropic properties of various agents are examined by measuring the aqueous solubility of paclitaxel. Paclitaxel is obtained from Samyang Genex Corp. (Taejeon, South Korea). The concentration of paclitaxel is determined by an isocratic reverse-phase HPLC (Agilent 1100 series, Agilent Technologies, Wilmington, Del.) using a Symmetry column (Waters Corporation, Milford, Mass.) at 25° C. The mobile phase consists of acetonitrile-water (45:55 v/v) with a flow rate of 1.0 ml/min. A diode array detector is set at 227 nm and linked to ChemStation software for data analysis. The paclitaxel concentrations in the samples are obtained from a calibration curve.

TABLE 2
Paclitaxel (PTX) solubilities in the presence of various hydrotropic agents¹

| | PTX Solubility | Standard |
|---|---|---|
| Hydrotropic agent (concentration used) | (mg/ml) | Deviation |
| None (PTX solubility in pure water) | 0.0003 | |
| N,N-diethylnicotinamide (3.5 M) | 39.071 | 0.600 |
| N-picolylnicotinamide (3.5 M) | 29.435 | 1.205 |
| N-allylnicotinamide (3.5 M) | 14.184 | 0.385 |
| Sodium salicylate (3.5 M) | 5.542 | 0.514 |
| 2-methacryloyloxyethyl phosphoryicholine (2.9 M) | 3.199 | 0.037 |
| Resorcinol (3.5 M) | 2.009 | 0.012 |
| N,N-dimethylnicotinamide (3.5 M) | 1.771 | 0.026 |
| N-methylnicotinamide (3.5 M) | 1.344 | 0.006 |
| Butylurea (3.5 M) | 1.341 | 0.071 |
| Pyrogallol (3.5 M) | 1.282 | 0.008 |
| 3-picolylacetamide (3.5 M) | 1.084 | 0.003 |
| Procaine HCl (2.5 M) | 0.720 | 0.005 |
| Nicotinamide (3.5 M) | 0.694 | 0.031 |
| Pyridine (3.5 M) | 0.658 | 0.080 |
| 3-picolylamine (3.5 M) | 0.552 | 0.063 |
| Sodium ibuprofen (1.5 M) | 0.500 | 0.070 |
| Sodium xylenesulfonate (2.5 M) | 0.481 | 0.080 |
| Ethyl carbamate (3.5M) | 0.300 | 0.028 |
| 6-Hydroxy-N,N-diethylnicotinamide (2.0 M) | 0.241 | 0.004 |
| Sodium p-toluenesulfonate (2.5 M) | 0.220 | 0.002 |
| Pyridoxal hydrochloride (2.5 M) | 0.216 | 0.008 |
| 1-Methyl-2-pyrrolidone (3.5 M) | 0.071 | 0.002 |
| Sodium benzoate (2.0 M) | 0.050 | 0.006 |
| 2-Pyrrolidone (3.5 M) | 0.038 | 0.002 |
| Ethylurea (3.5 M) | 0.030 | 0.003 |
| N,N-dimethylacetamide (3.5 M) | 0.015 | 0.002 |
| N-methylacetamide (3.5 M) | 0.012 | 0.001 |
| Isoniazid (1.0 M) | 0.009 | 0.002 |

¹Another 32 agents showed paclitaxel solubilities of 0.005 mg/ml (or 5 μg/ml) or less. They are, in descending order of solubilizing effect: nipecotamide (3.5 M), citric acid (2.0 M), sodium gentisate (1.0 M), N-isopropylacrylamide (1.5 M), methylurea (3.5 M), 1,3-diamino-2-hydroxypropane-N,N,N′,N′-tetramethylacetate (3.0 M), thiourea (2.5 M), 1-methylnicotinamide iodide (1.0 M), α-cyclodextrin (0.15 M), sodium thiocyanate
(8.6 M), urea (6.0 M), caffeine (0.1 M), glyceryl triacetate (0.2 M), glycerin (3.5 M), adenosine (0.005 M), γ-cyclodextrin (0.17 M), β-cyclodextrin (0.02 M), diisopropylnicotinamide (0.05 M), pyridine-3-sulfonic acid (1.0 M), o-benzoic acid sulfimide (0.01 M), 2,6-pyridinedicarboxamide (0.0025 M), 3,4-pyridinedicarboxamide (0.025 M), 4-aminosalicylic acid (0.005 M), L-tryptophan (0.05 M), salicylaldoxime (0.1 M), sucrose (2.0 M), L-lysine
(2.0 M), 4-aminobenzoic acid sodium salt (2.5 M), D-sorbitol (3.0 M), sodium L-ascorbate (3.0 M), sodium propionate (3.5 M), sodium acetate (4.0 M), 2-hydroxy-N,N-diethylnicotinamide (0.2 M), 2-hydroxy-3-picolylnicotinamide (0.0035 M), and 6-hydroxy-3-picolylnicotinamide (0.08 M).

The aqueous solubility of paclitaxel, as determined by high-pressure liquid chromatography (HPLC), is 0.3 μg/ml. Thus, a paclitaxel concentration of 0.3 mg/ml indicates a 1,000-fold increase in aqueous solubility. As shown in Table 2, the paclitaxel solubility was increased almost to 40 mg/ml by 3.5 M of N,N-diethylnicotinamide, which corresponds to more than a 100,000-fold increase in solubility. Table 2 clearly identifies a number of hydrotropic agents effective for increasing the water solubility of paclitaxel. Specifically, the hydrotropic agents that increase paclitaxel solubility in excess of 0.3 mg/ml are N,N-diethylnicotinamide, N-picolylnicotinamide, N-allylnicotinamide, sodium salicylate, 2-methacryloyloxyethyl phosphorylcholine, resorcinol, N,N-dimethylnicotinamide, N-methylnicotinamide, butylurea, pyrogallol, 3-picolylacetamide, procaine HCl, nicotinamide, pyridine, 3-picolylamine, sodium ibuprofen, sodium xylenesulfonate, and ethyl carbamate.

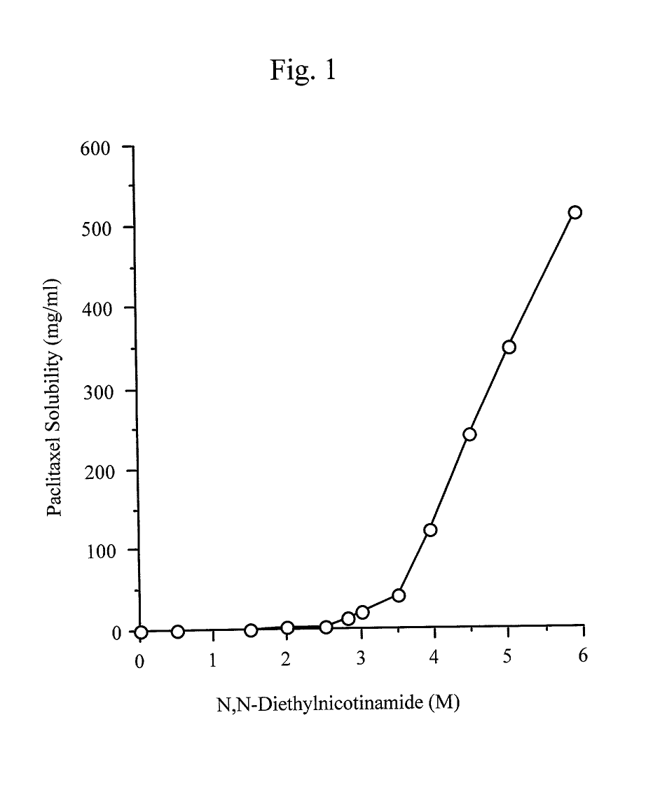

Of these, N,N-diethylnicotinamide was the best hydrotropic agent identified for increasing the water solubility of paclitaxel. N,N-diethylnicotinamide at 5.95 M increased the paclitaxel concentration to 512 mg/ml, which corresponds to about 10 N,N-diethylnicotinamide molecules for every paclitaxel molecule. The paclitaxel solubility as a function of N,N-diethylnicotinamide concentration is shown in FIG. 1.

B. Considerations for Rational Design/Selection of Hydrotropic Agents

Without wishing to be bound to any particular theory, it is surmised that the efficacy of a hydrotropic agent in enhancing the water solubility of a pharmaceutical compound depends on suitably matching the structural features of the hydrotropic agent with those of the drug. Accordingly, the structural characteristics of the hydrotropic agents listed in Table 2 were examined, viz., the structural features of paclitaxel. The chemical structure of paclitaxel is shown below:

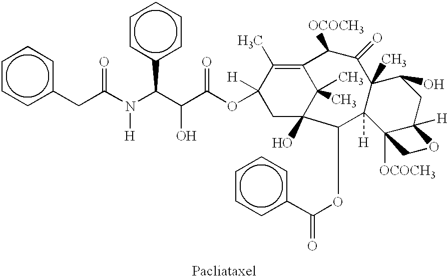

1. High Water-Solubility

The main criterion for effective hydrotropy is high water solubility of the hydrotropic agent. If the water solubility is low (e.g., less than 2 M), the hydrotropic properties are not significant. The agents that did not show any appreciable hydrotropic properties (discussed above for Table 2) also have poor water-solubilities. Examples are 4-aminosalicylic acid (0.005 M), salicylaldoxime (0.1 M), o-benzoic acid sulfimide (0.01 M), adenosine (0.005 M), glyceryl triacetate (0.2 M), caffeine (0.1 M), 2,6-pyridinedicarboxamide (0.0025 M), and 3,4-pyridinedicarboxamide (0.025 M). Those agents have low water solubility, and thus, almost no hydrotropic effect. The following examples show the importance of water solubility of hydrotropic agents on increasing aqueous paclitaxel (PTX) solubility.

| | PTX solubility | |
|---|---|---|
| Hydrotropic agent (concentration used) | (mg/ml) | Chemical structure |
| Nicotinamide (3.5 M) | 0.694 | 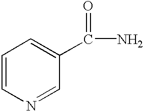 |
| 2,6-pyridinedicarboxamide (0.0025 M)* | 0.000 | 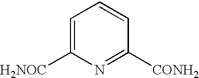 |
| 3,4-pyridinedicarboxamide (0.025 M)* | 0.000 | 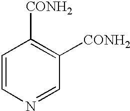 |

*The concentrations of 0.0025 M and 0.025 M are the maximum solubilities of these agents.

The importance of water solubility for derivatives of N,N-diethylnicotinamide is illustrated in the table below.

| | PTX solubility | |
|---|---|---|
| Hydrotropic agent (concentration used) | (mg/ml) | Chemical structure |
| N,N-diethylnicotinamide (3.5 M) | 39.071 | 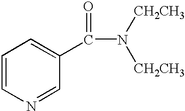 |
| 6-hydroxy-N,N-diethylnicotinamide (2.0 M)* | 0.241 | 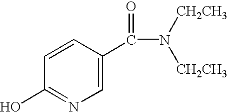 |
| 2-hydroxy-N,N-diethylnicotinamide (0.2 M)* | 0.000 | 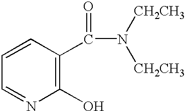 |

*The concentrations of 2.0 M and 0.2 M are the maximum solubilities of these agents.

b 2. High Hydrophobicity

For those agents having high water solubilities, the hydrotropic property increases as the hydrophobicity of the molecule increases. Poorly soluble organic drugs are hydrophobic and do not interact appreciably with water molecules through hydrogen bonding. Thus, the presence (or insertion) of hydrophobic drug molecules in water (known as hydrophobic hydration) causes a direct perturbation of water, i.e., an alteration in the hydrogen bonding state of water molecules. Since water is a condensed phase and each molecule possesses a finite volume, the hydrophobic molecules are excluded from the aqueous phase. This is known as the excluded volume effect, which is responsible for the poor water solubility of nonpolar compounds. (Graziano, G. 2000) Water structure formers, such as sucrose and sorbitol, inhibit dissolution of poorly soluble drugs, while water structure disruptors, such as nicotinamide, increase the solubility by destroying clusters of associated water molecules and releasing water of solvation (Müller, B. W. et al. 1991). Thus, effective hydrotropic agents are those that destabilize water structure and at the same time interact with poorly soluble drugs. Hydrophilic agents lacking a significant hydrophobic component are not effective at all. Examples are D-sorbitol (3.0 M), sucrose (2.0 M), citric acid (2.0 M), sodium L-ascorbate (3.0 M), L-lysine (2.0 M), sodium propionate (3.5 M), and sodium acetate (4.0 M). The following examples show the importance of hydrophobic groups in promoting hydrotropic properties.

2a. Pyridine and Aromatic Rings:

The most effective hydrophobic agents identified thus far contain pyridine and benzene rings. Almost all highly effective hydrotropic agents listed in Table 2 have either a pyridine ring or a benzene ring in their structures. Molecules without such rings in their structures generally are not as effective as molecules containing them. Nicotinamide and 3-picolylamine afforded about the same in paclitaxel solubility increase, while the hydrotropic property of nipecotamide (3.5 M), which has a saturated ring structure, is less than 1% that of nicotinamide (3.5 M). Similarly, urea (3.5 M), glycerin (3.5 M), thiourea (2.5 M), methylurea (3.5 M), N-isopropylacrylamide (1.5 M), N-methylacetamide (3.5 M), N,N-dimethylacetamide (3.5 M), and sodium thiocyanate (3.5 M) have very small hydrotropic effects. 1,3-diamino-2-hydroxypropane-N,N,N′,N′-tetramethylacetate (3.0 M) also showed poor hydrotropic properties.

| | PTX solubility | |
|---|---|---|
| Hydrotropic agent (concentration used) | (mg/ml) | Chemical structure |
| Nicotinamide (3.5 M) | 0.694 | 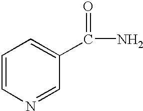 |
| 3-picolylamine (3.5 M) | 0.552 | 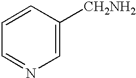 |
| Nipecotamide (3.5 M) | 0.005 | 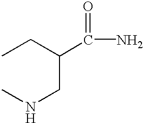 |
| N,N-dimethylacetamide (3.5 M) | 0.015 | 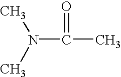 |
| N-isopropylacrylamide (1.5 M) | 0.004 | 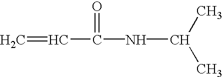 |
| 1,3-diamino-2-hydroxypropane- N,N,N′,N′-tetramethylacetate (3.0 M) | 0.004 | 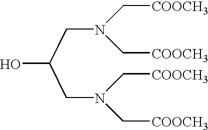 |

2b. Maximum Hydrophobicity Without Losing Water Solubility:

The hydrotropic properties of nicotinamide derivatives show a positive correlation with the molecule's hydrophobicity as long as water solubility is not lost. Thus, N,N-diethylnicotinamide shows more than a 20 times higher hydrotropic property than N,N-dimethylnicotinamide at the same concentration (3.5 M). N,N-dimethylnicotinamide, in turn, is more effective than N-methylnicotinamide and N-methylnicotinamide is twice more effective than nicotinamide. 1-Methylnicotinamide iodide is too hydrophilic to be hydrotropic. The poor hydrotropic properties of N,N-diisopropylnicotinamide are rationalized as being due to its poor water-solubility, which is only 0.05 M.

| | PTX solubility | |
|---|---|---|
| Hydrotropic agent (concentration used) | (mg/ml) | Chemical structure |
| N,N-diethylnicotinamide (3.5 M) | 39.07 | 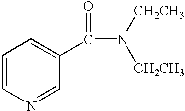 |
| N,N-dimethylnicotinamide (3.5 M) | 1.771 | 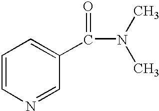 |
| N-methylnicotinamide (3.5 M) | 1.344 | 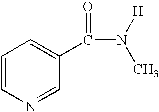 |
| Nicotinamide (3.5 M) | 0.694 | 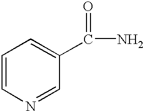 |
| 1-methylnicotinamide iodide (1.0 M)* | 0.003 | 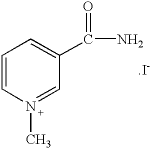 |
| N,N-diisopropylnicotinamide (0.05 M)* | 0.001 | 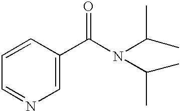 |

*The concentrations of 1.0 M and 0.05 M are the maximum solubilities of the agents.

2c. A Methyl Grou on the Ring Increases the Hydrotropic Property by a Factor of 2:

At the same concentration, sodium xylenesulfonate is more hydrotropic than sodium p-toluenesulfonate. A similar trend is seen with 1-methyl-2-pyrrolidone and 2-pyrrolidone. In both examples, the presence of one methyl group increases the “hydrotropicity” of the molecule by a factor of 2. The same result is observed for N-methylnicotinamide and nicotinamide.

| | PTX solubility | |
|---|---|---|
| Hydrotropic agent (concentration used) | (mg/ml) | Chemical structure |
| Sodium xylenesulfonate (2.5 M)* Sodium p-toluenesulfonate (2.5 M)* | 0.481 0.220 | 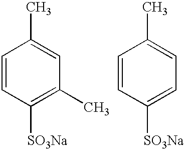 |
| 1-methyl-2-pyrrolidone (3.5 M) | 0.071 | 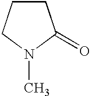 |
| 2-Pyrrolidone (3.5 M) | 0.038 | 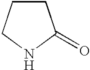 |
| N-methylnicotinamide (3.5 M) | 1.344 | 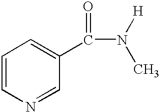 |
| Nicotinamide (3.5 M) | 0.694 | 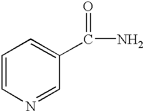 |

*The concentration of 2.5 M is the maximum solubility of the agent.

2d. One Long Hydrophobic Chain is More Effective Than Two Shorter Hydrophobic Chains:

As shown in the following table, the high hydrotropic properties of N-picolylnicotinamide and N-allylnicotinamide suggest that one longer carbon chain is better than two shorter carbon chains, e.g., one allyl group vs. two methyl groups.

| | PTX solubility | |
|---|---|---|
| Hydrotropic agent (concentration used) | (mg/ml) | Chemical structure |
| N-picolylnicotinamide (3.5 M) | 29.435 | 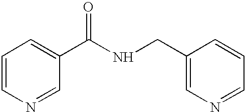 |
| N-allylnicotinamide (3.5 M) | 14.184 | 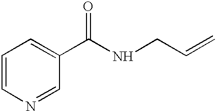 |
| N,N-dimethylnicotinamide (3.5 M) | 1.771 | 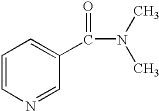 |

2e. Hydrotropic Agent Interaction With Solute:

Aliphatic derivatives of urea were studied for their effects on increasing the water solubility of paclitaxel. Butylurea shows the highest solubilizing effect of the analogs studied, which suggests that as the hydrophobicity decreases, the hydrotropic property also decreases. Urea is known to break up the hydrogen-bonded water molecule clusters surrounding nonpolar solute molecules. This leads to an increase in entropy favoring solubilization of hydrophobic molecules. (Martin, A. et al. 1993) The poor hydrotropic properties of neat urea suggests that disruption of water structure alone, without substantial interaction with solute, is not enough for effective hydrotropy.

| Hydrotropic agent | PTX solubility | |
|---|---|---|
| (concentration used) | (mg/ml) | Chemical structure |
| Butylurea (3.5 M) | 1.341 | 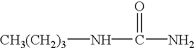 |
| Ethylurea (3.5 M) | 0.030 | 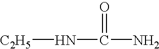 |
| Methylurea (3.5 M) | 0.004 | 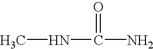 |
| Urea (3.5 M) | 0.001 | 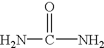 |

2f. Hydrotropic Properties are Reduced by an Increase in Hydrophiliciy:

A molecule's hydrophilicity can be increased by attaching hydroxyl groups to the molecule. This is observed to reduce the molecule's hydrotropic properties. Thus, resorcinol, which is more hydrophobic than pyrogallol, has better hydrotropic properties. Also studied was sodium gentisate, which has a lower water-solubility than the other two compounds, which limits its hydrotropic property.

| Hydrotropic agent | PTX solubility | |
|---|---|---|
| (concentration used) | (mg/ml) | Chemical structure |
| Resorcinol (3.5 M) | 2.009 | 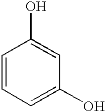 |
| Pyrogallol (3.5 M) | 1.282 | 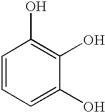 |
| Sodium gentisate (1.0 M) | 0.005 | 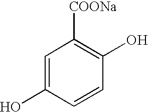 |

3. Separation of Hydrophilic and Hydrophobic Segments

Better hydrotropic agents are observed to have a clear separation between the hydrophilic and hydrophobic segments of the molecule. This is reasonable since hydrotropic agents are expected to have nonbonded hydrophobic interactions with hydrophobic solute molecules. It is interesting to note that sodium salicylate is highly effective in dissolving paclitaxel. Sodium salicylate (3.5 M), sodium ibuprofen (1.5 M), sodium xylenesulfonate (2.5 M), and sodium p-toluenesulfonate (2.5 M) show clear separation of hydrophilic and hydrophobic parts. The clear separation of hydrophilic and hydrophobic segments may make it possible to interact efficiently with hydrophobic solutes, such as paclitaxel. Sodium salicylate is well known for its ability to inhibit the self-association (usually through stacking) of hydrophobic molecules. (Martin, A. et al 1993) Similarly, 2-methacryloyloxyethyl phosphorylcholine (2.88 M) shows excellent hydrotropic propertes, which may be due to the clear separation of its hydrophilic and hydrophobic segments.

| | PTX solubility | |
|---|---|---|
| Hydrotropic agent (concentration used) | (mg/ml) | Chemical structure |
| Sodium salicylate (3.5 M) Sodium salicylate (2.5 M) | 5.542 0.912 | 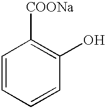 |
| Procaine·HCl (2.5 M) | 0.720 | 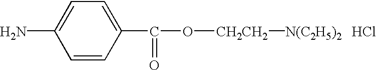 |
| Pyridine (3.5 M) | 0.658 | 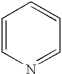 |
| Sodium ibuprofen (1.5 M) | 0.500 | 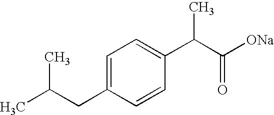 |
| Sodium xylenesulfonate (2.5 M) | 0.481 | 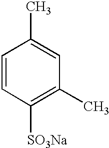 |
| Sodium p-toluenesulfonate (2.5 M) | 0.220 | 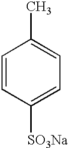 |
| Pyridoxal hydrochloride (2.5 M) | 0.216 | 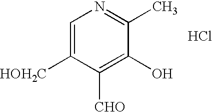 |
| Sodium benzoate (2.0 M) | 0.050 | 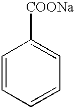 |
| Isoniazid (1.0 M) | 0.009 | 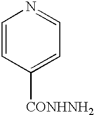 |
| Sodium gentisate (1.0 M) | 0.005 | 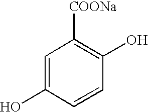 |
| Pyridine-3-sulfonic acid (1.0 M) | 0.001 | 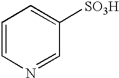 |
| 4-aminobenzoic acid sodium salt (2.5 M) | 0.000 | 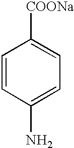 |
| 2-methacryloyloxyethyl phosphorylcholine (2.88 M) | 3.199 | 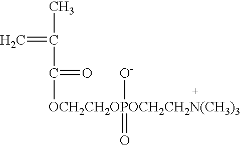 |

4. Other Low Molecular Weight Hydrotropic Agents

Based on the structures of hydrotropic agents identified in Table 2, one can synthesize more derivatives and other compounds having good hydrotropic properties for paclitaxel and other poorly soluble drugs. Since N,N-diethylnicotinamide, picolylnicotinamide, and salicylic acid showed good hydrotropic properties, derivatives of those compounds are also expected to be good hydrotropic agents with respect to a given drug compound. For example, derivatives of N,N-diethylnicotinamide that can increase the hydrotropic properties of the molecule include 6-hydroxy (or methoxy, or benzyloxy)-N,N-diethylnicotinamide, 2-acetamidomethyl (or aminomethyl)-N,N-diethylnicotinamide, and 3-nicotinamidomethyl-N,N-diethylnicotinamide. Picolylnicotinamide derivatives that can increase its hydrotropic properties include 6-hydroxy-2-picolylnicotinamide, 6-methoxy-3-picolylnicotinamide, and 6-benzyloxy-4-picolylnicotinamide. Derivatives of salicylic acid can include 3-aminosalicylic acid and 4-benzylaminosalicylic acid.

5. Increased Solubility of Other Poorly Soluble Drugs by Hydrotropic Agents

The two best hydrotropic agents studied for paclitaxel listed in Table 2 were N,N-diethylnicotinamide and N-picolylnicotinamide. These compounds were also used to examine the solubility increase of other poorly soluble drugs. The other poorly soluble drugs examined were griseofulvin, progesterone, and tamoxifen. Their chemical structures are shown below:

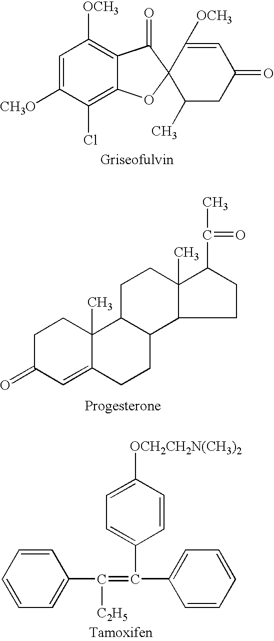

As listed in Table 1, the partition coefficients of griseofulvin, progesterone and tamoxifen are 2.07, 3.84, and 4.90, respectively. The water solubilities of these drugs vary from 0.4 μg/ml (similar to that of paclitaxel) to 7.0 μg/ml, while the partition coefficient ranges from 2.07 (lower than that of paclitaxel) to 4.90, which is an order of magnitude higher than paclitaxel. Table 2 presents the hydrotropic properties of N,N-diethylnicotinamide and picolylnicotinamide, viz., paclitaxel. The hydrotropic effects of both agents on griseofulvin, progesterone and tamoxifen were not as great as with paclitaxel, but the increase in aqueous solubilities was more than three orders of magnitude. Clearly, as shown in Table 3, the hydrotropic properties of N,N-diethylnicotinamide and picolylnicotinamide were highly effective—not only for paclitaxel but for other poorly soluble drugs as well.

TABLE 3
Aqueous solubilities of poorly soluble
drugs in the presence of various
hydrotropic agents.

| | Drug | Standard |
|---|---|---|
| Hydrotropic agent (concentration used) | (mg/ml) | Deviation |
| Griseofulvin | | |
| N,N-diethylnicotinamide | | |
| (0 M) (Control in pure water) | 0.007 | 0.000 |
| (0.5 M) | 0.044 | 0.000 |
| (1.0 M) | 0.268 | 0.003 |
| (3.5 M) | 9.750 | 0.191 |
| Picolylnicotinamide | | |
| (0 M) (Control in pure water) | 0.007 | 0.000 |
| (0.5 M) | 0.196 | 0.010 |
| (1.0 M) | 0.610 | 0.009 |
| (3.5 M) | 5.036 | 0.034 |
| Progesterone | | |
| N,N-diethylnicotinamide | | |
| (0 M) (Control in pure water) | 0.0012 | 0.0000 |
| (0.5 M) | 0.059 | 0.001 |
| (1.0 M) | 0.218 | 0.003 |
| (3.5 M) | 4.534 | 0.022 |
| Picolylnicotinamide | | |
| (0 M) (Control in pure water) | 0.0012 | 0.0000 |
| (0.5 M) | 0.514 | 0.019 |
| (1.0 M) | 1.296 | 0.016 |
| (3.5 M) | 14.275 | 0.166 |
| Tamoxifen | | |
| N,N-diethylnicotinamide | | |
| (0 M) (Control in pure water) | 0.0004 | 0.0000 |
| (0.5 M) | 0.002 | 0.000 |
| (1.0 M) | 0187 | 0.006 |
| (3.5 M) | 3.142 | 0.098 |
| Picolylnicotinamide | | |
| (0 M) (Control in pure water) | 0.0004 | 0.0000 |
| (0.5 M) | 0.002 | 0.000 |
| (1.0 M) | 0.014 | 0.000 |
| (3.5 M) | 1.595 | 0.020 |

II. Hydrotropic Polymers

Although many of the hydrotropic agents identified in Table 2 are considered safe and some have been used in humans, the use of rather high concentrations of the hydrotropic agents may pose a difficulty in formulation of drug delivery systems. This is mainly due to the possibility of absorption of a hydrotropic agent itself from the dosage form into the body, such as from the GI tract into the bloodstream. For this reason, it is desirable to identify polymeric hydrotropic agents that will not be absorbed from the GI tract, e.g., due to their extremely large molecular sizes. The hydrotropic polymers and copolymers are sometimes referred to herein as “hytrops.”

A. Synthesis of Hydrotropic Polymers.

Table 4 lists some of the hydrotropic polymers that have been synthesized based on the molecular structures of hydrotropic agents identified in Table 2.

TABLE 4
Exemplary hydrotropic polymers synthesized from hydrotropic agents.

Poly(6-(4-vinylbenzyloxy)-N-picolylnicotinamide 2HCl)
Poly(2-(4-vinylbenzyloxy)-N-picolylnicotinamide 2HCl
Poly(6-(4-vinylbenzyloxy)-N-picolylnicotinamide 2HCl-co-4-vinylpyridine HCl)
Poly(6-allyloxy-N-picolylnicotinamide 2HCl)
Poly(N-allylnicotinamide)
Poly(vinylbenzyltrimethyl ammonium chloride)
Poly(6-allyloxy-N,N-diethylnicotinamide)
Poly(sodium 6-allyloxynicotinic acid)
Poly(2-methacryloyloxyethyl phosphorylcholine-co-N-isopropylacrylamide)
Poly(Sodium 4-acrylamidosalicylate)
Poly(Sodium 5-acrylamidosalicylate)

An example of the synthesis of a hydrotropic polymer from an identified hydrotropic agent is described below for poly(6-(4-vinylbenzyloxy)-N-picolylnicotinamide) as a model hydrotropic polymer having an aromatic spacer group.

### EXAMPLE II-1

### Synthesis of Poly(6-(4-Vinylbenzyloxy)-N-Picolylnicotinamide)

The overall synthetic route for poly(6-(4-vinylbenzyloxy)-N-picolylnicotinamide) is shown below.

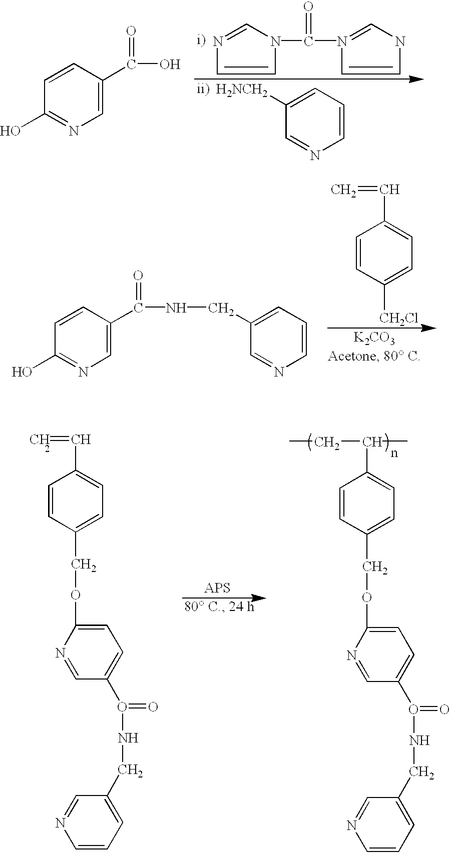

An analogous route can be used to synthesize poly(2-(4-vinylbenzyloxy)-N-picolylnicotinamide):

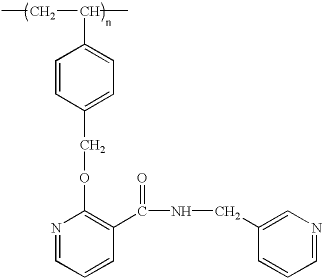

### EXAMPLE II-2

### Preparation of N-Picolylnicotinamide:

To a solution of 3-picolylnicotinamide (1.08 g, 10 mmol) and pyridine (1.58 g, 20 mmol) in dry methylene chloride (30 mL) is added nicotinoyl chloride hydrochloride (1.78 g, 10 mmol) at 0° C. The reaction mixture is stirred at room temperature for 24 h under nitrogen. After the end of reaction, the solvent is removed under reduced pressure, and the crude product is dissolved in water, neutralized with NaHCO₃, and extracted with chloroform. The solution is dried over anhydrous magnesium sulfate. The solvent is removed at reduced pressure, and the product is isolated by column chromatography on a silica gel using methylene chloride:methanol (98:2 v/v %). (Yield:80%)

### EXAMPLE II-3

### Synthesis of 6-Hydroxy-N-Picolylnicotinamide (6-HPNA):

6-HPNA is prepared following a one-pot two-step synthetic procedure. To a stirred suspension of 6-hydroxynicotinic acid (15 g, 0.108 mol) in THF (600 mL) is added 1,1′-carbonyldiimidazole (17.48 g, 0.108 mol) in one portion. The reaction mixture is stirred at reflux under nitrogen. After 24 h, 3-picolylamine (23.32 g, 0.216 mol) is added dropwise to the stirred suspension of N-(6-hydroxynicotinyl)-imidazole in THF at reflux. The reaction is maintained for 24 h under nitrogen. After cooling the reaction mixture to room temperature, the pale yellow precipitate is filtered, washed with diethyl ether, and dried in vacuo to yield 6-HPNA (Yield: 85%).

### EXAMPLE II-4

### Synthesis of 6-(4-Vinylbenzyloxy)-N-Picolylnicotinamide (6-VBOPNA):

A suspension of 6-HPNA (9g, 0.039 mol) and K₂CO₃ (13.57 g, 0.098 mol) in dry acetone is heated to 70° C. 4-Vinylbenzyl chloride (12 g, 0.079 mol) is then added dropwise to the reaction mixture. The reaction is maintained for 24 h under nitrogen. After the end of this period, the crude reaction mixture is filtered to obtain a thick brown liquid. The product 6-VBOPNA is isolated by column chromatography with n-hexane:THF (1:3 v/v %) on a silica gel. Yield: 70%.

### EXAMPLE II-5

### Synthesis of Poly(6-(4-Vinylbenzyloxy)-N-Picolylnicotinamide)) (P(6-VBOPNA)):

To a solution of 6-VBOPNA-2HCl (1.5 g, 3.6 mmol) with concentration of 1.0 M in distilled water, APS (8.3 mg, 0.04 mmol) is added. The mixture is degassed with a stream of nitrogen for 15 min. The reaction mixture is maintained for 24 h at 80 ° C. under nitrogen. At the end of this period, the polymer is isolated by dialysis using a membrane (Spectrapor, MWCO: 1000) against 6 L distilled water. The solution of P(6-VBOPNA.2HCl) is then dried at 60° C. in vacuo. (Yield: 53%)

### EXAMPLE II-6

### Synthesis of Poly(N-Allyl Nicotinamide):

N-allyl nicotinamide was polymerized by free radical polymerization using AIBN as an initiator. Other types of initiators can also be used.

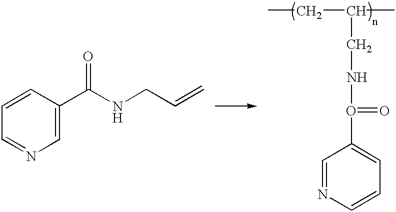

### EXAMPLE II-7

### Synthesis of 6-O-Acetylnicotinic Acid:

To a solution of 6-hydroxy nicotinic acid (25 mmol, 3.5g) in dry pyridine (10 ml) was added acetic anhydride (10 ml) and stirred at room temperature for 20 h (or until it turns into a clear solution). At the end of this period the solvent was removed by rotary evaporation and the brown solid (6-O-acetylnicotinic acid) thus obtained was dissolved in CHCl₃ (25 ml) and washed with water (2×10 ml) to remove acetic acid present. This was followed by rotary evaporation to obtain a brown solid which was purified by column chromatography over silica gel using CH₂Cl₂:MeOH (95:5%, v/v).

### EXAMPLE II-8

### Synthesis of 6-O-Acetylnicotinamide:

To a solution of 6-O-acetylnicotinic acid (10 mmol, 1.71 g) dissolved in dry CHCl₃ (2 0 ml) was added oxalylchloride (12 mmol, 1 ml) and stirred at room temperature for 24 h. At the end of this period at 0° C. ammonia solution was added dropwise (causing vigorous reaction) and stirred at room temperature for 2 h. The solvent was removed by rotary evaporation and the solid thus obtained was purified by column chromatography over silica gel using CH₂Cl₂:MeOH (98:2% v/v) as eluent.

### EXAMPLE II-9

### Synthesis of 6Hydroxynicotinamide:

To a solution of 6-O-acetylnicotinamide (10 mmol, 1.7 g) in THF (20 ml) was added 1 M NaOH (1 ml) added and stirred for 5 h at room temperature. At the end of this period the reaction mixture was acidified to pH 7 by the dropwise addition of diluted HCl. The white solid thus obtained was washed with water and used up for next step.

### EXAMPLE II-10

### Synthesis of 6-O-Acryloylnicotinamide:

To a solution of 6-hydroxynicotinamide (10 mmol, 1.38 g) in dry CH₂Cl₂ (20 ml) was added acryloyl chloride (11 mmol, 0.8 ml) under N₂ and continued stirring for 20 h. At the end of this period the solvent was removed by rotary evaporation and washed with NaHCO₃ solution (10 ml) and extracted with CHCl₃ and the solvent was removed in vacuo. The solid obtained was purified by column chromatography over silica gel using CH₂Cl₂:MeOH (98:2% v/v).

### EXAMPLE II-11

### Synthesis of Poly(6-Acryloylnicotinamide):

To a solution of 6-O-acryloylnicotinamide (5 mmol, 0.92 g) in DMF (20 ml) was added AIBN (0.02 mmol %) and refluxed at 70° C. for 20 h. The solvent was evaporated and the viscous solid was purified by washing with CH₂Cl₂ (30 ml).

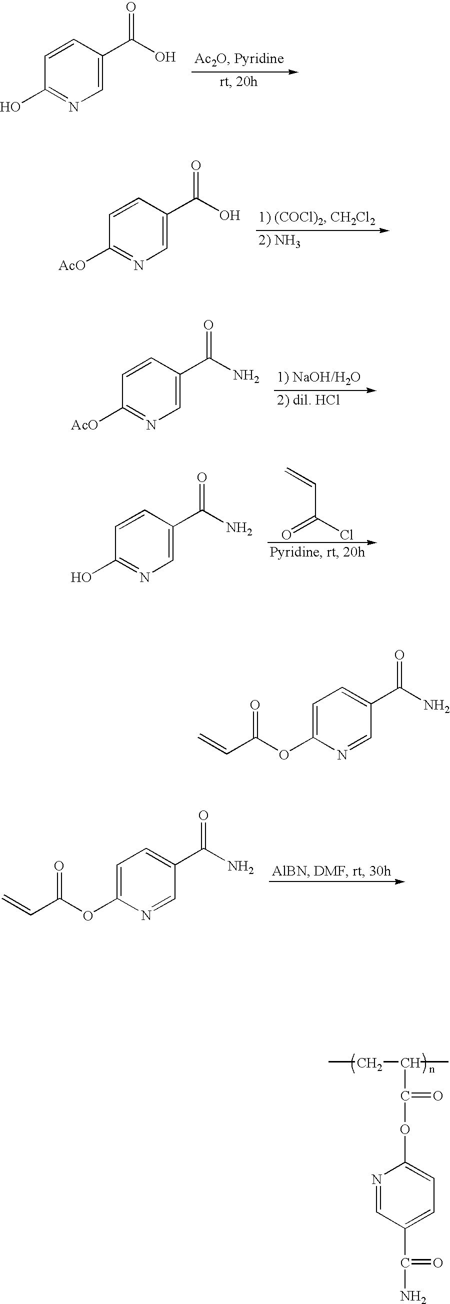

### EXAMPLE II-12

### Synthesis of 6-O-Acetyl-N,N-Dimethylylnicotinamide:

To a solution of 6-O-acetylnicotinic acid (10 mmol, 1.71 g) dissolved in dry CHCl₃ (20 ml) was added oxalylchloride (12 mmol, 1 ml) and stirred at room temperature for 24 h. At the end of this period at 0° C. N,N-dimethylamine in THF (20 ml ) was added dropwise (vigorous reaction occurs) and stirred at room temperature for 2 h. The solvent was removed by rotary evaporation and the solid thus obtained was purified by column chromatography over silica gel using CH₂Cl₂:MeOH (98:2% v/v) as eluent.

### EXAMPLE II-13

### Synthesis of 6-Hydroxy-N,N-Dimethylnicotinamide:

To a solution of 6-O-acetyl-N,N-dimethylnicotinamide (10 mmol, 1.98 g) in THF (20 ml) was added IM NaOH (1 ml) and stirred for 5 h at room temperature. At the end of this period the reaction mixture was acidified to pH 7 by the dropwise addition of diluted HCl. The white solid thus obtained was washed with water and used up for the next step.

### EXAMPLE II-14

### Synthesis of 6-O-Acryloyl-N,N-Dimethylnicotinamide:

To a solution of 6-hydroxy-N,N-dimethylnicotinamide (10 mmol, 1.57 g) in dry CH₂Cl₂ (20 ml) was added acryloyl chloride (11 mmol, 0.8 ml) under N₂ and continued stirring for 6 h. At the end of this period the solvent was removed by rotary evaporation and washed with NaHCO₃ solution (10 ml) and extracted with CHCl₃ and the solvent was removed in vacuo. The solid obtained was purified by column chromatography over silica gel using CH₂Cl₂:MeOH (98:2% v/v).

### EXAMPLE II-15

### Synthesis of Poly(6-Acryloyl-N,N-Dimethylnicotinamide):

To a solution of 6-O-acryloyl nicotinamide (5 mmol, 1.1 5 g) in DMF (20 ml) was added AIBN (0.2 mmol %) and refluxed at 70° C. for 20 h. The solvent was evaporated and the viscous solid was purified by washing with CH₂Cl₂ (30 ml).

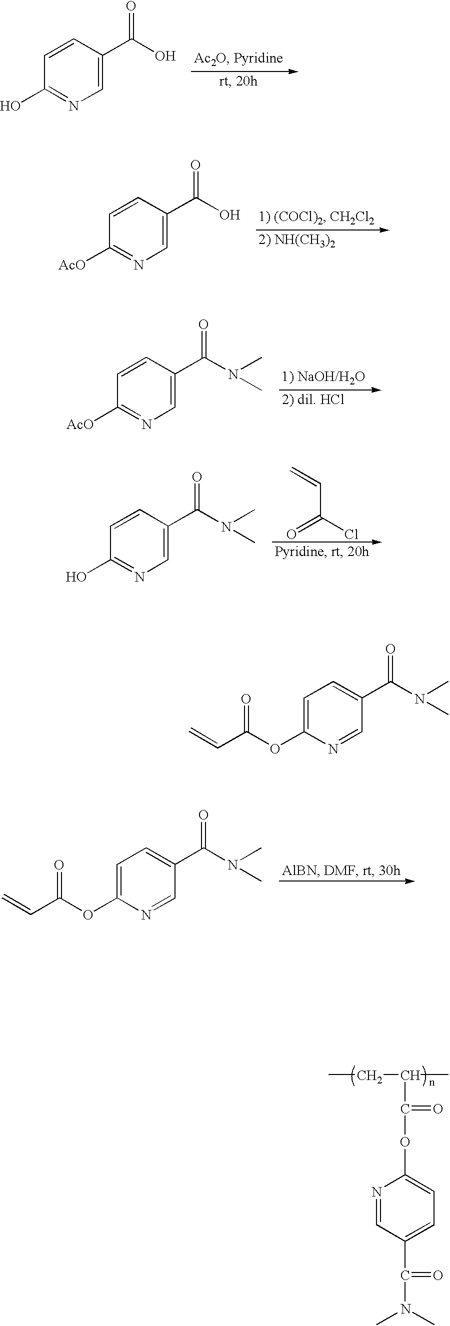

### EXAMPLE II-16

### Synthesis of 6-O-Acetyl-N,N-diethylnicotinamide:

To a solution of 6-O-acetylnicotinic acid (10 mmol, 1.71 g) dissolved in dry CHCl₃ (20 ml) was added oxalylchloride (12 mmol, 1 ml) and stirred at room temperature for 24 h. At the end of this period at 0° C. N,N-diethylamine (12mmol, 1.3 ml) was added dropwise (causing vigorous reaction) and stirred at room temperature for 2 h. The solvent was removed by rotary evaporation and the solid thus obtained was purified by column chromatography over silica gel using CH₂Cl₂:MeOH (98:2% v/v) as eluent.

### EXAMPLE II-17

### Synthesis of 6-Hydroxy-N,N-Diethylnicotinamide:

To a solution of 6-O-acetyl-N,N-diethylnicotinamide (10 mmol, 2.26 g ) in THF (20 ml) was added 1 M NaOH (1 ml) added and stirred for 5 h at room temperature . At the end of this period the reaction mixture was acidified to pH 7 by the dropwise addition of diluted HCl. The white solid thus obtained was washed with water and used up for next step.

### EXAMPLE II-18

### Synthesis of 6-O-Acryloyl-N,N-Diethylnicotinamide:

To a solution of 6-hydroxy-N,N-diethylnicotinamide (10 mmol, 1.85 g) in dry CH₂Cl₂ (20 ml) was added acryloyl chloride (11 mmol, 0.8 ml) under N₂ and continued stirring for 6 h. At the end of this period the solvent was removed by rotary evaporation and washed with NaHCO₃ solution (10 ml) and extracted with CHCl₃ and the solvent was removed in vacuo. The solid obtained was purified by column chromatography over silica gel using CH₂Cl₂:MeOH (98:2% v/v).

### EXAMPLE II-19

### Synthesis of Poly(6-O-Acryloyl-N,N-Diethylnicotinamide):

To a solution of 6-O-acryloyl-N,N-diethyl nicotinamide (5 mmol, 1.2 g) in DMF (20 ml) was added AIBN (0.2 mmol %) and refluxed at 70° C. for 20 h. The solvent was evaporated and the viscous solid was purified by washing with CH₂Cl₂ (30 ml).

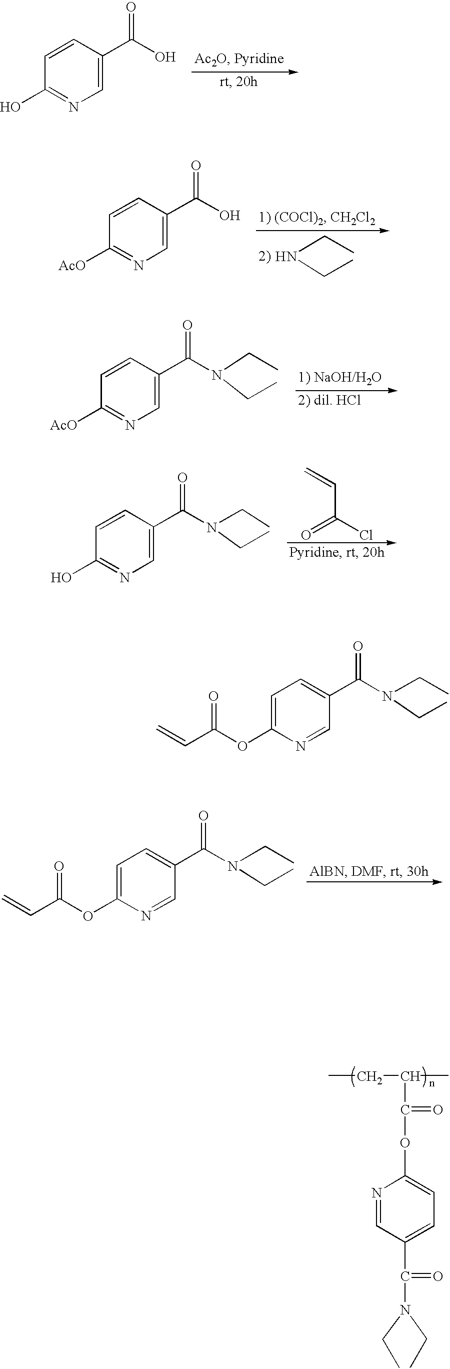

### EXAMPLE II-20

### Synthesis of 6-O-Acetyl-N-Picolylnicotinamide:

To a solution of 6-O-acetylnicotinic acid (10 mmol, 1.71 g) dissolved in dry CHCl₃ (20 ml) was added oxalylchloride (12 mmol, 1 ml) and stirred at room temperature for 24 h. At the end of this period at 0° C. picolylamine (12 mmol, 1.2 ml) was added dropwise (causing vigorous reaction) and stirred at room temperature for 2 h. The solvent was removed by rotary evaporation and the solid thus obtained was purified by column chromatography over silica gel using CH₂Cl₂:MeOH (98:2% v/v) as eluent.

### EXAMPLE II-21

### Synthesis of 6-Hydroxy-N-Picolylnicotinamide:

To a solution of 6-O-acetyl-N-picolyl nicotinamide (10 mmol, 2.61 g) in THF (20 ml) was added 1 M NaOH (1 ml) added and stirred for 5 h at room temperature. At the end of this period the reaction mixture was acidified to pH 7 by the dropwise addition of diluted HCl. The white solid thus obtained was washed with water and used up for next step.

### EXAMPLE II-22

### Synthesis of 6-O-Acryloyl-N-Picolylnicotinamide:

To a solution of 6-hydroxy-N-picolylnicotinamide (10 mmol, 2.2 g) in dry CH₂Cl₂ (20 ml) was added acryloyl chloride under N₂ and continued stirring for 6 h. At the end of this period the solvent was removed by rotary evaporation and washed with NaHCO₃ solution (10 ml) and extracted with CHCl₃ and the solvent was removed in vacuo. The solid obtained was purified by column chromatography over silica gel using CH₂Cl₂:MeOH (98:2% v/v).

### EXAMPLE II-23

### Synthesis of Poly(6-O-Acryloyl-N-Picolylnicotinamide):

To a solution of 6-O-acryloyl-N-picolylnicotinamide (5 mmol, 1.4 g) in DMF (20 ml) was added AIBN (0.2 mmol %) and refluxed at 70° C. for 20 h. The solvent was evaporated and the viscous solid purified by washing with CH₂Cl₂ (30 ml).

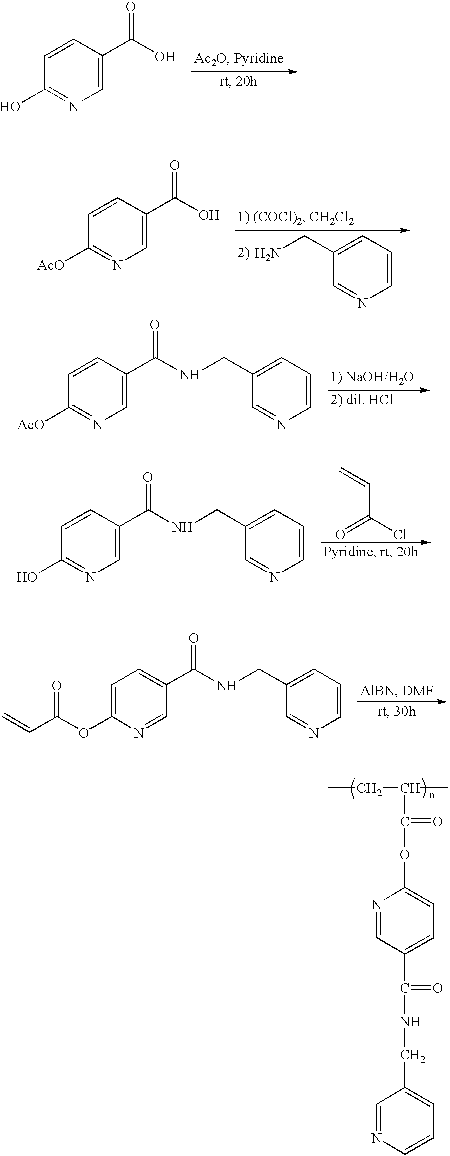

### EXAMPLE II-24

### Synthesis of 3-Pyridylacrylamide:

To a solution of 3-aminopyridine (10 mmol, 1 g) in dry CH₂Cl₂ (30 ml) at 0° C. was added acryloyl chloride (10 mmol, 0.32 ml) dropwise over a period of 15 min. After the addition was complete, the ice bath was removed and continued stirring for 6 h. At the end of this period, the solvent was removed by rotary evaporation to obtain a yellow solid. The solid thus obtained was dissolved in the minimum amount of water (10 ml) and neutralized with NaHCO₃ solution, followed by extraction with CHCl₃ (3×20 ml). The organic layer was dried over Na₂SO₄ and concentrated by rotary evaporation to obtain a yellow solid. The product was purified by column chromatography over silica gel using CH₂Cl₂: MeOH (98:2% v/v).

### EXAMPLE II-25

### Synthesis of Poly(3-Pyridylacrylamide):

To a solution of 3-pyridylacrylamide (10 mmol, 1.4 g) dissolved in DMF (20 ml) was added AIBN (0.2 mmol %) and stirred at 60° C. for 10 h. At the end of this period the solvent was removed by rotary evaporation and the solid thus obtained was washed with MeOH (3×25 ml) and dried under vacuum.

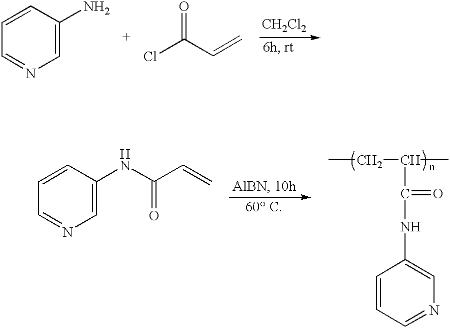

### EXAMPLE II-26

### Synthesis of Nicotinamide Polymer by Chemical Grafting:

The following reaction illustrates a route for grafting a nicotinamide moiety onto a preformed polyamine polymer by condensing an acid derivative of the nicotinamide with the polyamine. Polymers of other nicotinamide derivatives can be similarly prepared. The synthesis of polyesters by grafting can also be obtained by the corresponding condensation reactions between a polyol and acid monomer unit or poly(meth)acrylate and alcohol monomer unit. Such reactions are conventional and readily applied.

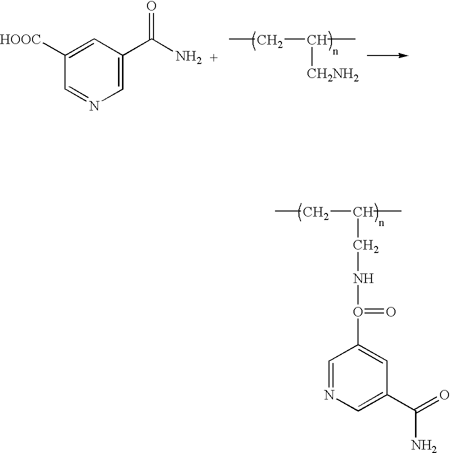

### EXAMPLE II-27

### Synthesis of Poly(6-(4-Vinylbenzyloxy)N,N-Diethylnicotinamide):

Polymers based on N,N-diethylnicotinamide can be prepared following a similar procedure as shown in the scheme below. The synthesis of poly(2-(4-vinylbenzyloxy)-N,N-diethynicotinamide) can be done by simply using 2-hydroxynicotinic acid instead of 6-hydroxynicotinic acid as a starting material.

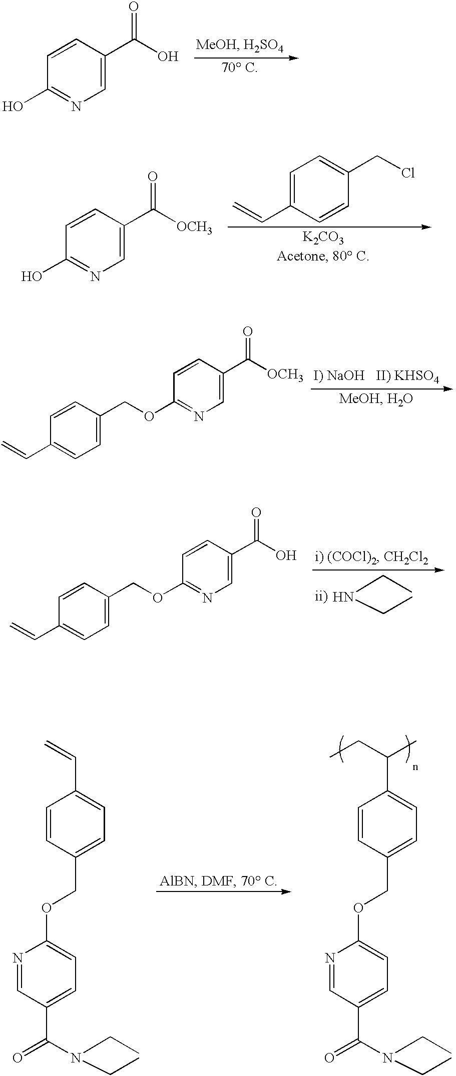

### EXAMPLE II-28

### Synthesis of Poly(Sodium 3-(4-Vinylbenzyl)Aminosalicylate):

Hydrotropic polymers possessing the sodium salicylate moiety are also synthesized with different orientations of the hydrotropic moiety. The reaction scheme is shown below for poly(sodium 3 -(4-vinylbenzyl)aminosalicylate. Poly(sodium 4-(4-vinylbenzyl) amino salicylate) and poly(sodium 5 -(4-vinylbenzyl)aminosalicylate) are synthesized following the same reaction scheme using 4-aminosalicylic acid and 5-aminosalicylic acid, respectively, in place of 3-aminosalicylic acid. The polymerizable monomers are synthesized through the reduction of each Schiff base.

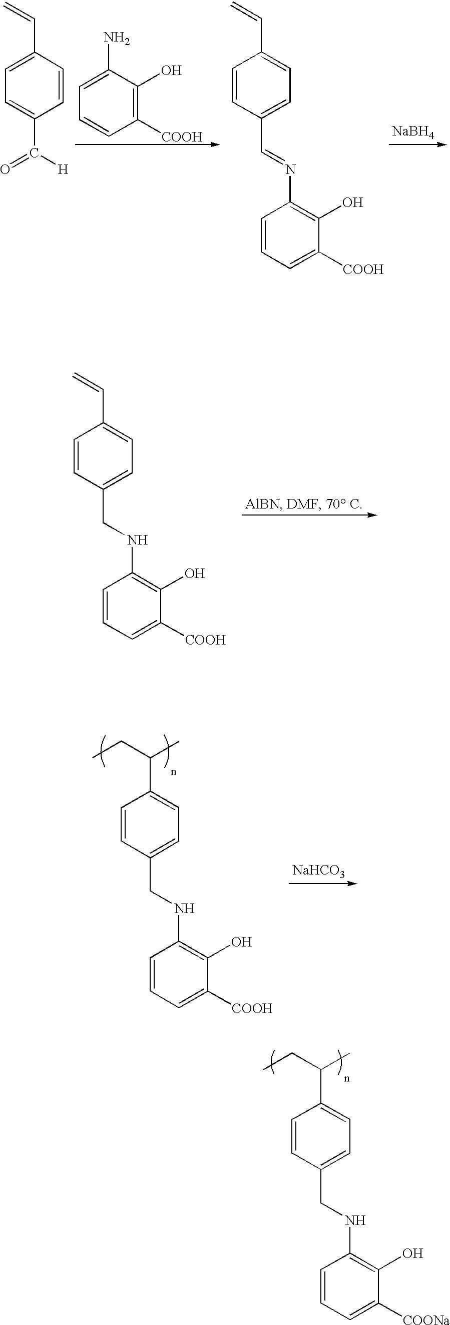

### EXAMPLE II-29

### Synthesis of Ethylene Glycol (EG) Spacer Compounds:

Hydrotropic polymers having EG spacers can also be synthesized. The length of the spacers is varied from 2 to 6 EG units. The synthesis of these polymers is based on the selective reaction of carbonyldiimidazole. It is expected that the longer the EG chains, the more rotation of the hydrotropic moieties, thereby leading to improved hydrotropic properties. Shown below, is a synthetic scheme for polymers having a sodium salicylate moiety bound to EG spacers at the 3-position. Other polymer structures having sodium salicylate moieties bound to EG spacers at 4- and 5-positions can be prepared similarly. Hydrotropic polymers based on N-picolylnicotinamide and N,N-diethylnicotinamide but provided with EG spacers can also be synthesized with the reactions outlined hereinabove.

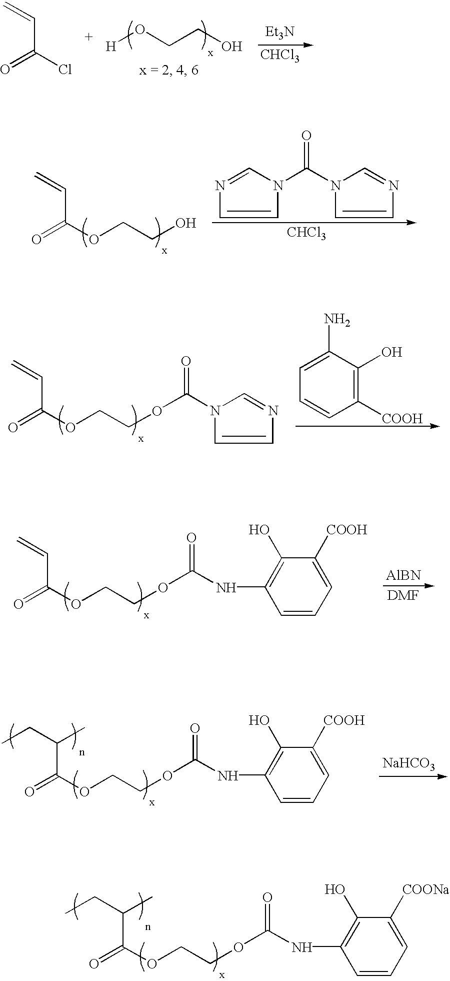

### EXAMPLE II-30

### Synthesis of Copolymers Having Different Orientations of the Same Hydrotropic Moiety:

Polymers containing the same hydrotropic moiety in different orientations are synthesized by copolymerization of monomers obtained from the same hydrotrope. This approach can provide an opportunity of the facile interaction of hydrotropic units with paclitaxel by compensating the motional limitation of each polymer-bound hydrotropic moiety. Hydrotropic copolymers having N-picolylnicotinamide, N,N-diethylnicotinamide, and sodium salicylate, which have different orientations to polymer backbone, can be synthesized. Examples of copolymers made of the same hydrotropic agent in different orientations having an aromatic spacer are shown below.

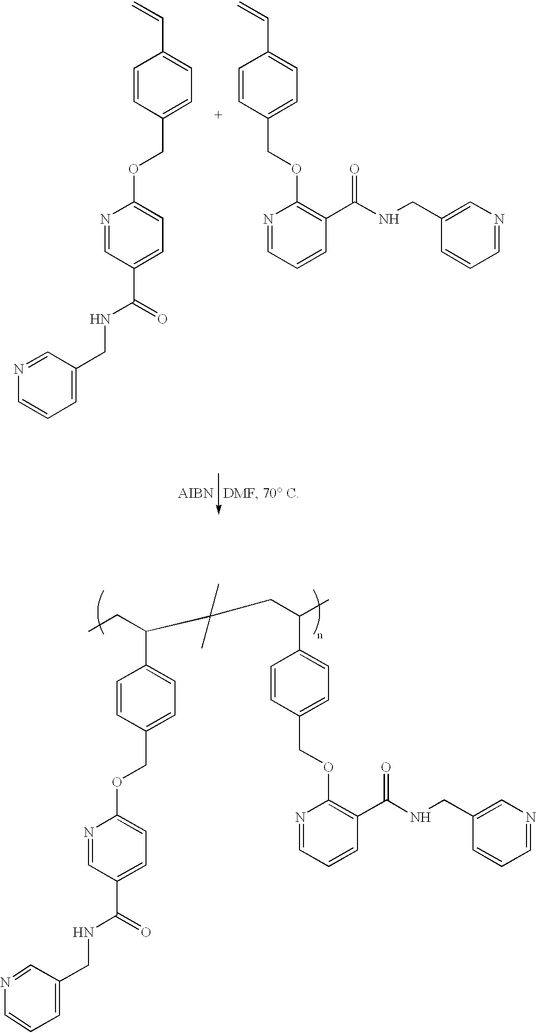

Synthesis of poly(6-(4-vinylbenzyloxy)-N-picolylnicotinamide-co-2-(4-vinylbenzyloxy)-N-picolylnicotinamide)

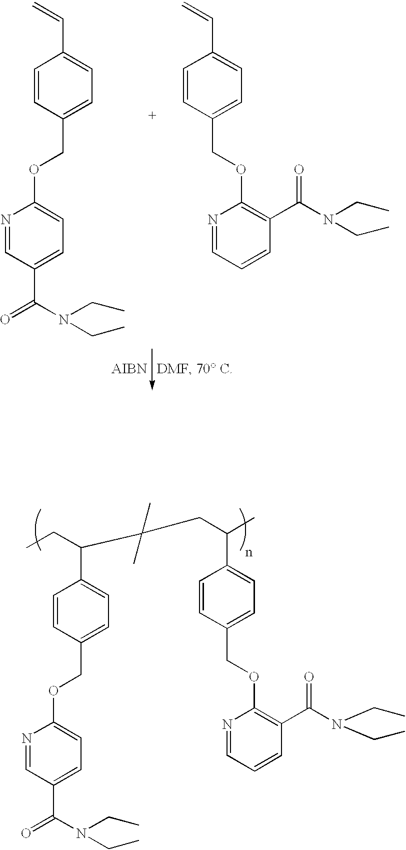

Synthesis of poly(6-(4-vinylbenzyloxy)-N,N-diethylnicotinamide-co-2-(4-vinylbenzyloxy)-N,N-diethylnicotinamide)

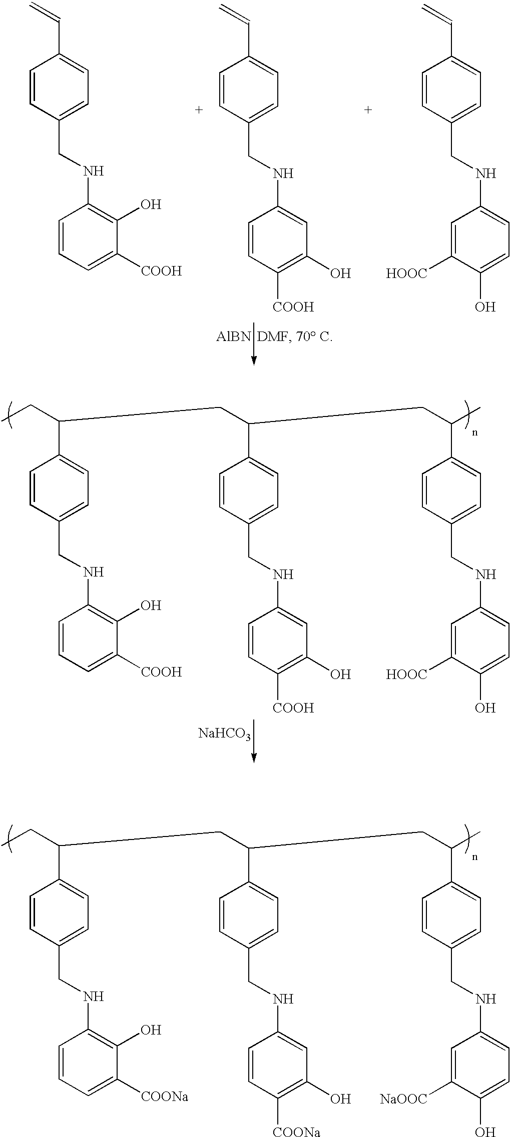

Synthesis of poly(sodium 3-(4-vinylbenzyl)aminosalicylate-co-4-(4-vinylbenzyl)aminosalicylate-co-5-(4-vinylbenzyl)aminosalicylate)

### EXAMPLE II-31

### Synthesis of Copolymers Having EG Spacers:

As shown below, copolymers of hydrotropic agents having EG spacers between the polymer backbone and the hydrotropic moieties can be synthesized. The synthesis of sodium salicylate-based hydrotropic copolymers having EG spacer units between the polymer backbone and hydrotropic moieties is shown. Again, the number of EG units is varied from 2 to 6. Where the hydrotropic moiety is attached in three different orientations, it may be advantageous if the length of the EG units is different for each orientation. It may provide more space among the dangling hydrotropic moieties in different orientations.

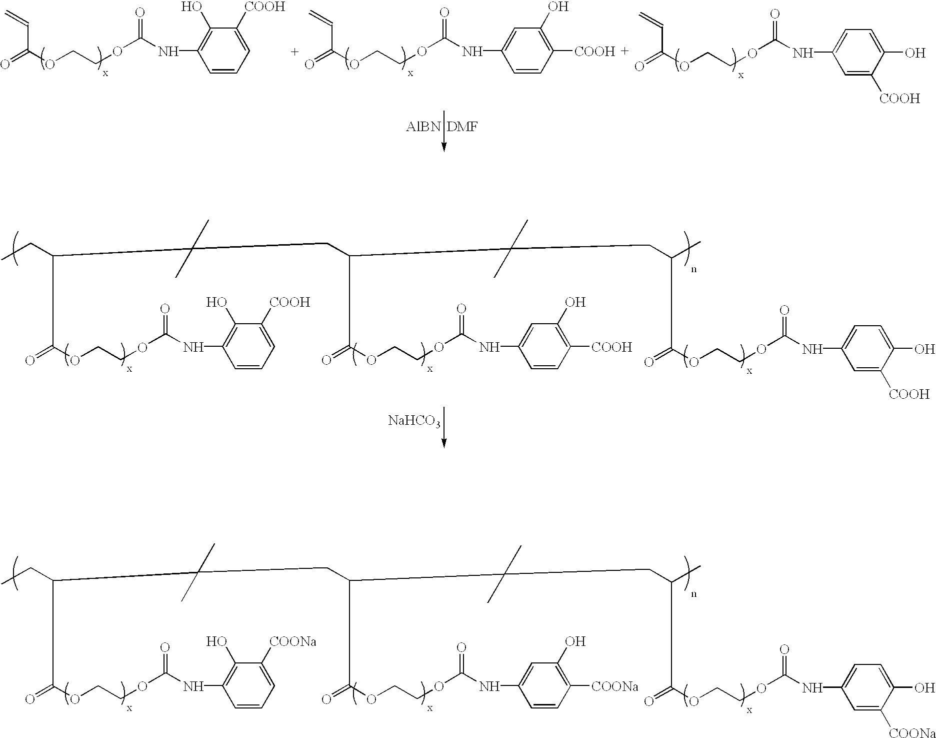

B. Hydrotropic Properties of the Newly Synthesized Polymers

The hydrotropic effects of the above-mentioned newly synthesized polymers were tested and the results are listed in Table 5 hereinbelow. At the bottom of Table 5 are also listed two polymers, polyethyleneglycol (PEG) and polyvinylpyrrolidone (PVP), which have been frequently used in the preparation of solid dispersions of poorly soluble drugs. (Habib, M. J. et al. 2001). PEG at 50% concentration is able to dissolve paclitaxel at a concentration of 0.133 mg/ml. PVP, on the other hand, did not show any appreciable hydrotropic property for paclitaxel. The concentrations of PVP could not go higher than 20% due to increased viscosity of the solution.

A number of hydrotropic polymers were synthesized based on picolylnicotinamide, N,N-diethylnicotinamide, pyridine, allylnicotinamide, and sodium salicylate. These polymers showed a paclitaxel solubility in the range of 0.1 mg/ml to 1 mg/ml. In Table 5, even 2% poly(6-(4-vinylbenzyloxy)-N-picolylnicotinamide.2HCl) showed 0.152 mg/ml solubility of paclitaxel. This is more than 500 times higher paclitaxel solubility than in pure water. Use of the hydrotropic polymer is limited by an increase in viscosity of the solution, which suggests that the use of low molecular weight polymers should increase the hydrotropic properties even more. The potential for further improvements is quite promising.

As described herein, most highly effective hydrotropic agents for paclitaxel contain either a pyridine or an aromatic ring. The aromaticity of the pyridine and the aromatic rings may be the most important contributor to the solubilization, e.g., by the promotion of stacking of molecules through their planarity. Therefore, hydrotropic copolymers are prepared by increasing the content of pyridine and/or aromatic rings. The copolymers of 4-vinylpyridine with monomers based on N-picolylnicotinamide and N,N-diethylnicotinamide are synthesized. The copolymers of monomers having aromatic ring and sodium salicylate-based monomers are also synthesized.

Synthesized polymers are characterized by analysis of NMR spectra. ¹H NMR and ¹³C NMR spectra are obtained on a Bruker ARX 300 spectrometer. Molecular weights and molecular weight distributions are determined using a gel permeation chromatography equipped with an Agilent 1100 series RI detector, quaternary pump, and PL aquagel-OH columns with pore sizes of 30 Å, 40 Å, and 50 Å. The eluent is water, and the molecular weights are calibrated with poly(ethyleneoxide) standards.

TABLE 5
Hydrotropic properties of hydrotropic polymers for paclitaxel (PTX)¹

| | PTX | Standard |
|---|---|---|
| Hydrotropic Polymer (concentration used) | (mg/ml) | Deviation |
| 6-(4-vinylbenzyloxy)-N-picolylnicotinamide2HCl (monomer control) | | |
| (98%, 2.34 M) | 3.033 | 0.067 |
| (57%, 1.36 M) | 1.320 | 0.024 |
| (37.6%, 0.90 M) | 0.616 | 0.007 |
| (20%, 0.48 M) | 0.212 | 0.010 |
| (15%, 0.36 M) | 0.109 | 0.000 |
| (10%, 0.24 M) | 0.037 | 0.003 |
| (5%, 0.12 M) | 0.001 | 0.000 |
| (4%, 0.10 M) | 0.001 | 0.000 |
| (2%, 0.05 M) | 0.001 | 0.000 |
| Poly(6-(4-vinylbenzyloxy)-N-picolylnicotinamide 2HCl) | | |
| (98%, 2.34 M) | 1.146 | 0.058 |
| (57%, 1.36 M) | 0.912 | 0.048 |
| (37.6%, 0.90 M) | 0.883 | 0.092 |
| (10%, 0.24 M) | 0.457 | 0.005 |
| (4%, 0.10 M) | 0.308 | 0.026 |
| (2%, 0.05 M) | 0.152 | 0.014 |
| 2-(4-vinylbenzyloxy)-N-picolylnicotinamide 2HCl | | |
| (22.9%, 0.66 M) (monomer control) | 0.519 | — |
| Poly(2-(4-vinylbenzyloxy)-N-picolylnicotinamide 2HCl | | |
| (22.9%, 0.66 M) | 0.534 | 0.034 |
| Poly(6-(4-vinylbenzyloxy)-N-picolylnicotinamide 2HCl)-co-(4-vinylpyridine HCl) | | |
| (58.7%) | 0.368 | 0.002 |
| (29.4%) | 0.192 | — |
| (17.7%) | 0.152 | — |
| (3.5%) | 0.093 | — |
| 6-Allyloxy-N-picolylnicotinamide 2HCl) | | |
| (1.0 M) | 0.002 | 0.000 |
| (2.0 M) | 0.836 | 0.025 |
| Poly(6-allyloxy-N-picolylnicotinamide 2HCl) | | |
| (54%, 2.0 M) | 0.305 | 0.047 |
| N-Allylnicotinamide (36%, 2.2 M) | 2.364 | 0.007 |
| Poly(N-allylnicotinamide) (36%, 2.2 M) | 0.253 | 0.020 |
| Vinylbenzyltrimethyl ammonium chloride | | |
| (49.5%, 2.33 M) (monomer control) | 0.552 | 0.060 |
| (20.5%, 0.97 M) (monomer control) | 0.039 | 0.002 |
| Poly(vinylbenzyltrimethyl ammonium chloride) | | |
| (20.5%, 0.97 M) | 0.158 | 0.022 |
| 6-Allyloxy-N,N-diethylnicotinamide | | |
| (1.2 M) (monomer control) | 0.132 | 0.002 |
| Poly(6-allyloxy-N,N-diethylnicotinamide) | | |
| (27.2%, 1.2 M) | 0.149 | 0.003 |
| Poly(sodium 6-allyloxynicotinic acid) (18%, 1.0 M) | 0.003 | 0.001 |
| Poly(2-methacryloyloxyethyl phosphorylcholine-co-N-isopropylacrylamide) | | |
| (2%) | 0.042 | 0.022 |
| Poly(Sodium 4-acrylamidosalicylate) | | |
| (23.3%, 1.02 M) | 0.028 | 0.001 |
| Poly(Sodium 5-acrylamidosalicylate) | | |
| (23.3%, 1.02M) | 0.000 | 0.000 |
| (Polymers used in solid dispersions) | | |
| Poly(ethylene glycol) 400 (50%, 1.25 M) | 0.133 | 0.007 |
| Poly(ethylene glycol) 400 (30%, 0.75 M) | 0.001 | 0.000 |
| Poly(ethylene glycol) 400 (10%, 0.25 M) | 0.0004 | 0.0001 |
| Poly(ethylene glycol) 900 (50%, 0.56 M) | 0.089 | 0.002 |
| Poly(ethylene glycol) 2000 (50%, 0.25 M) | 0.087 | 0.004 |
| Poly(ethylene glycol) 200 (50%, 2.5 M) | 0.075 | 0.009 |
| Poly(ethylene glycol) 2000 (30%, 0.15 M) | 0.007 | 0.000 |
| Pluronic P85 (10%) | 0.118 | 0.007 |
| Pluronic F127 (10%) | 0.066 | 0.005 |
| Pluronic L61 (0.024%) | 0.000 | 0.000 |
| Polyvinylpyrrolidone K-25 | | |
| (10%, 0.003 M) & (20%, 0.006 M) | 0.003 | 0.001 |
| Polyvinylpyrrolidone K-90 (10%, 0.000077 M) | 0.002 | 0.000 |
| Polyvinylpyrrolidone K-30 (20%, 0.0034 M) | 0.001 | 0.000 |
| Polyvinylpyrrolidone K- 17 (20%, 0.025 M) | 0.000 | 0.000 |

¹The molar concentrations listed after the w/v % concentrations for homopolymers are the concentrations of monomers present in the polymers in order to compare the hydrotropic property of the polymers with that of low molecular weight counterparts.

C. Comparison of Hydrotropic Properties of Hydrotropic Agents and Polymers Thereof

In the absence of clearly understood mechanisms on how hydrotropic agents increase water solubility of poorly soluble drugs, it is difficult to predict a priori whether the corresponding hydrotropic polymers would be as effective as their monomers or low molecular weight counterparts. It has been suggested that the hydrotropic solubilization process involves cooperative intermolecular interactions with several balancing molecular forces, rather than either a specific complexation event or a process dominated by a medium effect, such as cosolvency or salting-in. (Tavare, N. S. et al. 1996; Dhara, D. et al. 1999) Thus, it is reasonable to assume that the hydrotropic molecules can have equal or better hydrotropic properties in a polymer form due to cooperative interactions with hydrophobic drugs than in a low molecular weight monomeric form.

1. Modification of Low Molecular Weight Hydrotropic Agents.

To synthesize a hydrotropic polymer, a hydrotropic agent usually needs to be modified to introduce a polymerizable moiety, such as a vinyl group. Introduction of a vinyl group to a hydrotropic agent typically results in an increase in its hydrotropic properties. For example, when N-picolylnicotinamide is modified to introduce a vinyl group, the monomeric form, 2-(4-vinylbenzyloxy)-N-picolylnicotinamide), shows more than an eight-fold increase in hydrotropic properties from 0.063 mg/ml to 0.519 mg/ml. Significantly, the hydrotropic properties of the monomer are maintained even after being polymerized into poly(2-(4-vinylbenzyloxy)-N-picolylnicotinamide).

| | PTX solubility | |
|---|---|---|
| Hydrotropic agent (concentration used) | (mg/ml) | Chemical structure |
| N-picolylnicotinamide (0.66 M) | 0.063 | 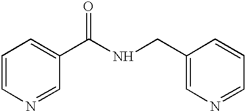 |
| 2-(4-vinylbenzyloxy)-N- picolylnicotinamide) (22.9%, 0.66 M) | 0.519 | 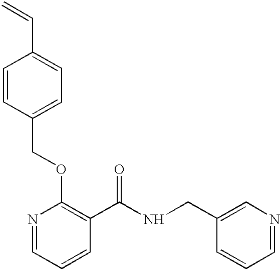 |
| Poly(2-(4-vinylbenzyloxy)-N- picolylnicotinamide) (22.9%, 0.66 M) | 0.534 | 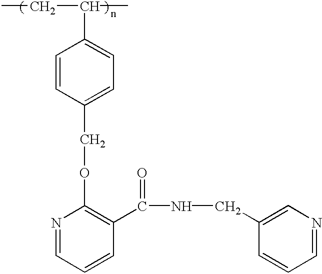 |

2. Concentration-Dependent Properties of Hydrotropic Polymers

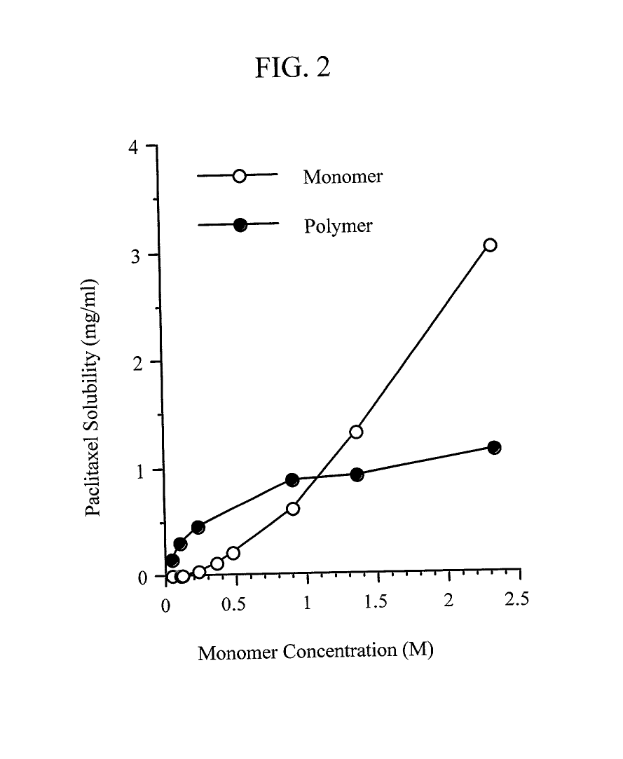

FIG. 2 shows the increase in paclitaxel solubility in the presence of monomeric and polymeric forms of 6-(4-vinylbenzyloxy)-N-picolylnicotinamide. It is noted that the polymer has better hydrotropic properties at concentrations of 1 M and lower. At concentrations higher than 1 M, the monomer showed better hydrotropic properties. Other hydrotropic polymers also showed the general trend that at lower concentrations the polymers showed better hydrotropic properties but vice versa at higher concentrations.

The following examples also support the observation that at concentrations lower than about 1 M, polymers show a better hydrotropic effect, but vice versa at higher concentrations.

The paclitaxel solubility using 0.66 M of 2-(4-vinylbenzyloxy)-N-picolylnicotinamide) was 0.519 mg/ml, but that using its polymer (at the same monomer concentration) was 0.534 mg/ml.

The paclitaxel solubility using 1.2 M of 6-allyloxy-N,N-diethylnicotinamide was 0.132 mg/ml, but that using its polymer at the same monomer concentration was 0.149 mg/ml.,

Vinylbenzyltrimethyl ammonium chloride gave a paclitaxel solubility of 0.039 mg/ml at 0.97 M, but its polymer, poly(vinylbenzyltrimethyl ammonium chloride), increased paclitaxel solubility to 0.158 mg/ml at the same monomer concentration.

Unlike the increase in paclitaxel solubility shown by the polymers listed above, a high paclitaxel solubility of 2.364 mg/ml using N-allylnicotinamide at 2.2 M was reduced to only 0.253 mg/ml using its polymer at the same monomer concentration.

The trend observed here is particularly significant because hydrotropic polymers are most useful at lower concentrations, approximately 1 M or lower. As the concentration of the polymer increases, it may not provide the same hydrotropic effect as the corresponding monomer due to a variety of reasons. For instance, the increase in viscosity may hinder rearrangement of the molecules for effective shielding of paclitaxel from water, and at higher polymer concentrations polymer chains may entangle reducing the overall efficacy. Therefore, it may be advantageous to control the molecular weight (chain length) of hydrotropic polymers so that the maximum hydrotropic effect is obtained at any concentration.

3. Role of Spacer Group Between Polymer Backbone and the Hydrotropic Moiety

While the structure of the hydrotropic moiety of the polymer is believed to be the most important factor in hydrotropy, other factors can contribute to the overall hydrotropic property of the polymers. The spacer group between the polymer backbone and the hydrotropic moiety may be one key factor affecting the overall hydrotropy. As shown in the following example, two different hydrotropic polymers based on N-picolylnicotinamide have different hydrotropic properties depending on the nature of the spacer. The paclitaxel solubility of poly(6-(4-vinylbenzyloxy)-N-picolylnicotinamide) was 0.883 mg/ml at the concentration of 0.90 M. When the aromatic spacer was replaced with a linear chain in poly(6-allyloxy-N-picolylnicotinamide), the paclitaxel solubility was only 0.305 mg/ml even when the concentration of the polymer was increased to 2.0 M. Therefore, as long as the spacer group does not negatively affect the water solubility of the polymer, a more hydrophobic spacer is desirable.

| Hydrotropic agent | PTX solubility | |
|---|---|---|
| (concentration used) | (mg/ml) | Chemical structure |
| N-picolylnicotinamide (0.90 M) | 0.227 | 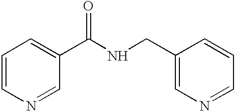 |
| Poly(6-(4-vinylbenzyloxy)-N-picolyl- nicotinamide) (37.6%, 0.90 M) | 0.883 | 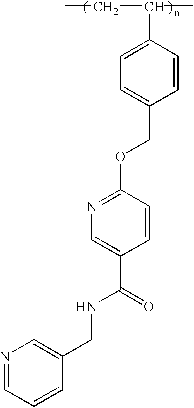 |
| Poly(6-allyloxy-N-picolyl- nicotinamide) (2.0 M) | 0.305 | 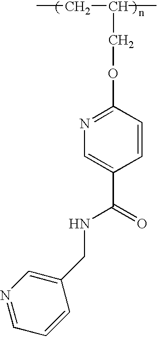 |

4. Variations of Hydrotropic Polymers

In addition to a spacer group, hydrotropic polymers can be made using the same hydrotropic moiety but with different orientations by copolymerization of different monomers obtained from the same hydrotrope. This approach can provide an opportunity for facile interaction of hydrotropic units with paclitaxel by compensating the motional limitation of each polymer-bound hydrotropic moiety. A copolymer having N-picolylnicotinamide at different orientations to the polymer backbone is shown below.

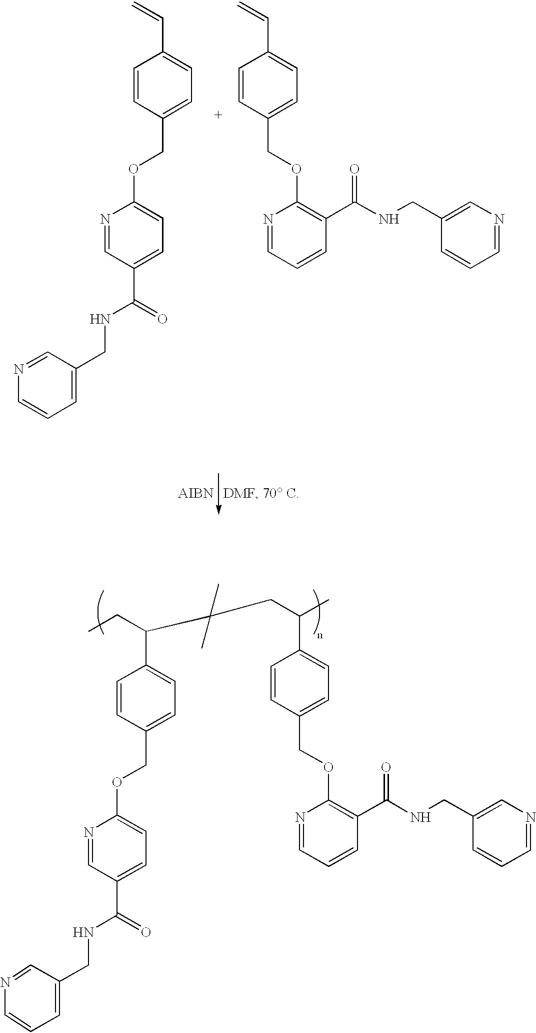

Poly(6-(4-vinylbenzyloxy)-N-picolylnicotinamide-co-2-(4-vinylbenzyloxy)-N-picolylnicotinamide).

Hydrotropic copolymers can also be made using two different hydrotropes. The concept of using two different hydrotropes on the same polymer backbone is based on the notion of “facilitated hydrotropy,” which involves the use of a combination of different hydrotropic agents to yield higher hydrotropic properties compared to the individual hydrotropes. (Yalkowsky, S. H. 1999) The maximum synergistic hydrotropic effect can be obtained by optimizing such factors as type and length of spacers, orientations of a hydrotrope, and the use of different hydrotropes.

5. Increased Solubility of Other Poorly Soluble Drugs by Hydrotropic Polymers

Increases in the water solubility of other poorly soluble drugs, such as griseofulvin, pregesteron, and tamoxifen, by employing hydrotropic polymers were measured using poly(2-(4-vinylbenzyloxy)-N-picolylnicotinamide2HCl (P(2-VBOPNA)). The monomeric form, 2-(4-vinylbenzyloxy)-N-picolylnicotinamide-2HCl (2-VBOPNA), and picolylnicotinamide (PNA) were also tested to compare the effect of hydrotropic polymers. As shown in the tables below, the monomeric unit (vinyl-containing) form of picolylnicotinamide was better than PNA itself, and the polymeric form was even better than the monomer. Clearly, hydrotropic polymers are superior to their monomeric counterparts, which opens up new possibilities of formulating a wide variety of poorly soluble drugs using hydrotropic polymers and hydrogels.

Griseofulvin solubility in hydrotropic solutions at 37° C. Mean ±SD, n=3.

| Concentration (M) | PNA | 2-VBOPNA | P(2-VBOPNA) |
|---|---|---|---|
| 0.0 | 0.007 ± 0.000 | 0.007 ± 0.000 | 0.007 ± 0.000 |
| 0.5 | 0.196 ± 0.010 | 0.343 ± 0.019 | 0.619 ± 0.014 |
| 1.0 | 0.610 ± 0.009 | 0.705 ± 0.026 | 0.987 ± 0.054 |

Progesterone solubility in hydrotropic solutions at 37° C. Mean ±SD, n=3.

| Concentration (M) | PNA | 2-VBOPNA | P(2-VBOPNA) |
|---|---|---|---|
| 0.0 | 0.0012 ± 0.0000 | 0.0012 ± 0.0000 | 0.0012 ± 0.0000 |
| 0.5 | 0.514 ± 0.019 | 0.683 ± 0.022 | 0.779 ± 0.044 |
| 1.0 | 1.296 ± 0.016 | 1.126 ± 0.041 | 1.322 ± 0.089 |

Tamoxifen solubility in hydrotropic solutions at 37° C. Mean ±SD, n=3.

| Concentration (M) | PNA | 2-VBOPNA | P(2-VBOPNA) |
|---|---|---|---|
| 0.0 | 0.00035 ± 0.00001 | 0.00035 ± 0.00001 | 0.00035 ± 0.00001 |
| 0.5 | 0.002 ± 0.000 | 0.603 ± 0.017 | 1.028 ± 0.025 |
| 1.0 | 0.014 ± 0.000 | 0.941 ± 0.046 | 1.733 ± 0.045 |

III. Hydrotropic Hydrogels (Hytrogels)

Hydrotropic hydrogels (sometimes referred to herein as “hytrogels”) can be prepared by chemically crosslinking one or more hydrotropic polymers as described hereinabove. This can be done by conducting crosslinking polymerization of hydrotropic agent monomers and/or by crosslinking of previously formed hydrotropic polymers. One of the advantages of hytrogels is that they provide a simple way of formulating poorly soluble drugs. Poorly soluble drugs can be loaded inside the hytrogels and the drug-loaded hytrogels can be used after drying. Since poorly soluble drugs are hydrophobic in nature, they are not expected to migrate to the surface of the hytrogel during drying and this minimizes or eliminates the burst release that is observed in most controlled release formulations.

Any of the hydrotropic polymers listed hereinabove can be made into hytrogels by simply adding a bifunctional crosslinking agent to the hydrotropic agent monomer solution. The following example illustrates the synthesis of a hytrogel based on 2-(4-vinylbenzyloxy)-N-picolylnicotinamide. A poorly soluble drug can be added to the monomer solution before polymerization or it can be loaded after the hytrogel is formed.

### EXAMPLE III-1

### Hytrogels Based on Poly(2-(4-Vinylbenzyloxy)-N-Picolylnicotinamide)

Paclitaxel (10 mg) is added to 1 ml aqueous solution of 2-(4-vinylbenzyloxy)-N-picolylnicotinamide.2HCl (2-VBOPNA). The concentration of 2-VBOPNA is taken either as 0.66 M or 1.2 M. The mixture is stirred vigorously and equilibrated for 24 h at 37° C. The 24 h equilibrium step can be skipped if excess paclitaxel is present. The paclitaxel/monomer suspension is filtered by passing it through a Millipore 0.2 μm filter. To the filtered solution is added ethylene glycol dimethacrylate, a crosslinker at a concentration of 6 mol % to the monomer. After degassing with dry nitrogen for 30 min, 2,2′-azobis(2-methylpropionamidine) dihydrochloride, a water-soluble initiator, is added at a concentration of 1 mol % to the monomer and the solution is placed in an oil bath at 60° C. The polymerization solution is maintained for 24 h. The resulting paclitaxel concentrations in the hytrogels made of 0.66 M and 1.2 M of 2-VBOPNA were 0.5 mg/ml and 1.2 mg/ml, respectively. As shown in the table below, the hydrotropic properties of the hytrogels are equivalent to those of the corresponding hydrotropic polymers. The hytrogels remain clear, which indicates that the loaded paclitaxel (PTX) is in the dissolved state.

| Hydrotropic agent | PTX Solubility |
|---|---|
| N-Picolylnicotinamide (0.66 M) | 0.063 mg/ml |
| 2-(4-vinylbenzyloxy)-N-picolylnicotinamide | 0.519 mg/ml |
| (0.66 M) | |
| Poly(2-(4-vinylbenzyloxy)-N-picolylnicotinamide) | 0.534 mg/ml |
| (0.66 M) | |
| Poly(2-(4-vinylbenzyloxy)-N-picolylnicotinamide) | 0.519 mg/ml |
| gel (0.66 M) | |

Paclitaxel can also be loaded into hytrogels after the hytrogel is formed. The synthesized hytrogels are purified by washing with copious amounts of water to remove any remaining initiator and crosslinking agent. The dried hytogel is swelled again in ethanol solution containing paclitaxel at various concentrations ranging from 0.5 mg/ml to 20 mg/ml.

### EXAMPLE III-2

### Hytrogels Based on 2-Methacryloyloxyethyl Phosphorylcholine

2-methacryloyloxyethyl phosphorylcholine (MPC) is dissolved in water to make a final concentration ranging from 20% to 85 (w/v) %. To the MPC solution is added ammonium persulfate (0.5% of MPC) and bisacrylamide (0.25, 0.5, 0.75, or 1.0% of MPC). The solution is kept at 60° C. and the MPC hytrogel is formed within 30 min.

In one approach, paclitaxel is dissolved directly into the monomer mixture to make a final concentration of 3 mg/ml before formation of the MPC hytrogel. The formed MPC hytrogel remains clear indicating the dissolved state of the loaded paclitaxel. In another approach, a hytrogel is formed first, washed with a copious amount of water and then dried at room temperature. The purified, dried hytrogel is placed into ethanol containing dissolved paclitaxel. Paclitaxel is loaded inside the MPC hytrogel after it swells in ethanol. The concentration of paclitaxel in ethanol varies up to 20 mg/ml.

IV. Preparation and Evaluation of Pharmaceutical Formulations

A pharmaceutical composition of the present invention contains a poorly soluble drug and a solubilizing compound, i.e., excipient, such as described hereinabove. Large molecular weight compounds are especially preferred excipients. Formulation of such compositions is illustrated hereinbelow for the case of paclitaxel, however, it is to be appreciated that methods and materials similar to these can be employed for other drugs.

The dosages of the drugs used in the present invention must, in the final analysis, be set by the physician in charge of the patient, using knowledge of the drugs, the properties of the drugs in combination as determined in clinical trials, and the characteristics of the patient, including diseases other than that under treatment by the physician. Only general outlines of the dosages are provided here.

Oral administration is not the only route or even the only preferred route, however. Other routes include transdermal, percutaneous, intravenous, intramuscular, intranasal, and intrarectal, in particular circumstances. The route of administration may be varied in any way, limited by the physical properties of the drugs and the convenience of the patient and the caregiver. The drug and excipient(s) can also be concurrently administered by more than one route.

It is particularly preferred, however, for a present formulation to be administered as a single pharmaceutical composition. Such compositions may take any physical form that is pharmaceutically acceptable, but orally usable pharmaceutical compositions are particularly preferred. Such pharmaceutical compositions contain an effective amount of each of the compounds, which effective amount is related to the daily dose of the compounds to be administered. Each dosage unit may contain the daily dose of one or more pharmaceutically effective drugs, or may contain a fraction of the daily doses, such as one-third of the doses. The amounts of each drug contained in each dosage unit depends on the identity of the drugs chosen for the therapy and other factors, such as the indication for which the therapy is being given.

The inert ingredients and manner of formulation of the pharmaceutical compositions are conventional, except for the presence of a solubility enhancing excipient as detailed within. The usual types of compositions may be used, including tablets, chewable tablets, capsules, solutions, parenteral solutions, intranasal sprays or powders, troches, suppositories, transdermal patches and suspensions. In general, compositions contain from about 0.1% to about 50% of the drug compounds in total, depending on the desired doses and the type of composition to be used. The amount of the compounds, however, is best defined as the effective amount, i.e., the amount of each compound that provides the desired dose to the patient in need of such treatment. The activity of the composition does not depend on its nature, therefore, the compositions are chosen and formulated solely for convenience and economy. Any of the combinations may be formulated in a desired form. Some discussion of different compositions follows.

Capsules are prepared by mixing the drug compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

Tablet disintegrants absorb water, swell, and break up the tablet, thereby releasing the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Tablets are often coated with sugar as a flavor and sealant, or with film-forming protecting agents to modify the dissolution properties of the tablet. The compounds may also be formulated as chewable tablets, by using large amounts of pleasant-tasting substances such as mannitol in the formulation. Instantly dissolving tablet-like formulations are also now frequently used to assure that the patient consumes the dosage form, and to avoid the difficulty in swallowing solid objects that bothers some patients.

A lubricant is necessary in a tablet formulation to prevent the tablet and punches from sticking in the die. The lubricant is chosen from such slippery solids as talc, magnesium and calcium stearate, stearic acid and hydrogenated vegetable oils.

Enteric formulations are often used to protect an active ingredient from the strongly acid contents of the stomach. Such formulations are created by coating a solid dosage form with a polymer film, which is insoluble in acid environments and soluble in basic environments. Exemplary films are cellulose acetate phthalate, polyvinyl acetate phthalate, hydroxypropyl methylcellulose phthalate and hydroxypropyl methylcellulose acetate succinate.

When it is desired to administer the combination as a suppository, the usual bases may be used. Cocoa butter is a traditional suppository base, which may be modified by addition of waxes to raise its melting point slightly. Water-miscible suppository bases comprising polyethylene glycols of various molecular weights can also be used.

Transdermal patches have become a popular route of administration recently. Typically they comprise a resinous composition in which the drugs will dissolve, or partially dissolve. The composition is held in contact with the skin by a film that protects it. More complicated patch compositions are also in use.

A. Preparation of Microparticles of Paclitaxel/Hydrotropic Polymer Formulations.

1. Current Commercial Paclitaxel Formulation

Paclitaxel is clinically proven active against advanced ovarian and breast cancer and is under investigation for various other types of cancers. The recommended doses for clinical applications of paclitaxel are 135 mg/m² and 175 mg/m² for small (1.4 m²) and large (2.4 m²) patients, respectively. These equal to the total paclitaxel quantities of 189 mg and 420 mg. The current clinical dosage form of paclitaxel consists of a 5 ml vial containing a total of 30 mg of paclitaxel, 2.635 g of Cremophor EL, and 49.7% ethanol (1:1 v/v), which is to be diluted with 0.9% sodium chloride or 5% dextrose injection solution to 0.3 mg/ml or 1.2 mg/ml before i.v. administration. Even with the use of Cremophor and ethanol, the total volume of the delivery solution is either 350 ml and 630 ml. If one uses pure water, then the delivery volumes would increase to 630 liters and 1,400 liters, which are physically impossible to deliver. The poor solubility has resulted in serious formulation problems, and this has also caused difficulties in other routes of delivery, such as oral administration. The presence of hydrotropic polymers is expected to eliminate the use of Cremophor EL, and ethanol in the paclitaxel formulation, lowering the toxicity of the current formulation significantly. The oral paclitaxel formulations using hydrotropic polymers are expected to increase the paclitaxel bioavailability due to the increased paclitaxel solubility in water.

2. Paclitaxel/Hydrotropic Polymer Formulations

Two different paclitaxel/hydrotropic polymer formulations are used herein to illustrate operation of the invention: liquid and solid formulations. Both formulations are used for in vitro cytotoxicity studies as well as animal experiments. These formulations are specifically for the proposed specific aims, and for this reason, the formulations are made as simple as possible.

The minimum effective concentration of paclitaxel is known to be 0.1 μmol/L, which is equivalent to approximately 0.1 μg/ml (0.1 μmol/L×854 g/mol=0.0854 μg/ml ˜0.1 μg/ml). The oral dose of the paclitaxel/hydrotropic polymer formulations are adjusted to obtain the blood paclitaxel concentration of 0.1 μg/ml and higher. A recent study done on oral administration of water-soluble paclitaxel derivatives used the oral dose of paclitaxel derivatives varying from 50 mg/kg to 200 mg/kg. Thus, the similar range of paclitaxel is employed in the beginning. The i.v. dose is varied from 10 mg/kg to 50 mg/kg.

The paclitaxel formulations are based on hydrotropic polymers, which, due to their large molecular weights, are not absorbed from the GI tract and remain on the surface of the GI tract to provide a continuous supply of paclitaxel.

Liquid Formulations

The liquid formulations are prepared by dissolving hydrotropic polymers in aqueous solution first and then dissolving paclitaxel to the desired concentrations. The liquid formulations are administered to rats through chronically implanted catheters, as described hereinbelow. The presence of chronic catheters allows administration of liquid dosage form, and the effect of a hydrotropic polymer formulation can be tested easily. This particular approach is useful since the administered hydrotropic polymer solution is not diluted much by the fluid present in the GI tract of the rats. Thus, the effect of high paclitaxel solubility in aqueous solution (1˜10 mg/ml and higher) on bioavailability can be tested. All aqueous solutions are prepared just before use.

Solid Formulations

Three types of solid formulations of paclitaxel/hydrotropic polymers are prepared.

The solid formulations allow long-term storage before use.

(1) Microspheres of paclitaxel and hydrotropic polymers are prepared by spray drying using a spray dryer (LAB-PLANT SD-05 from Scientific Instruments & Technology Corp.). The size of microspheres can be controlled between 1 μm to 30 μm. Slow dissolution of the microspheres in the GI tract provides high concentrations of the hydrotropic polymers in local regions and thus locally high paclitaxel concentrations.

(2) Loosely crosslinked hydrogel microspheres are prepared. This is to prepare for the situation where hydrotropic polymers dissolved from microspheres are diluted in the GI tract for any reason, thereby lowering the local concentration of the hydrotropic polymers. In this aspect, a crosslinking agent, such as N,N′-methylene-bis-acrylamide or diethylene glycol diacrylate, is added during polymerization of hydrotropic polymers. Once the hydrogel block is formed, it can be made into microspheres by simple grinding. Paclitaxel can be loaded into hydrogel microspheres by adding the dried microspheres into a water/acetonitrile mixture containing dissolved paclitaxel. The solubility of paclitaxel in acetonitrile is 200 mg/ml, and the concentration of the loaded paclitaxel can be controlled by adjusting the water/acetonitrile ratios. The paclitaxel-loaded hydrogel microspheres are dried until use. The hydrotropic hydrogel microspheres ensure that the hydrotropic polymers maintain a certain concentration as well as the solubility of the paclitaxel loaded inside the microspherical hydrogels. The paclitaxel release kinetics are controlled by adjusting a few parameters, such as the total amount of paclitaxel, the concentration and type of hydrotropic polymers, crosslinking density, and the total number of microspheres.

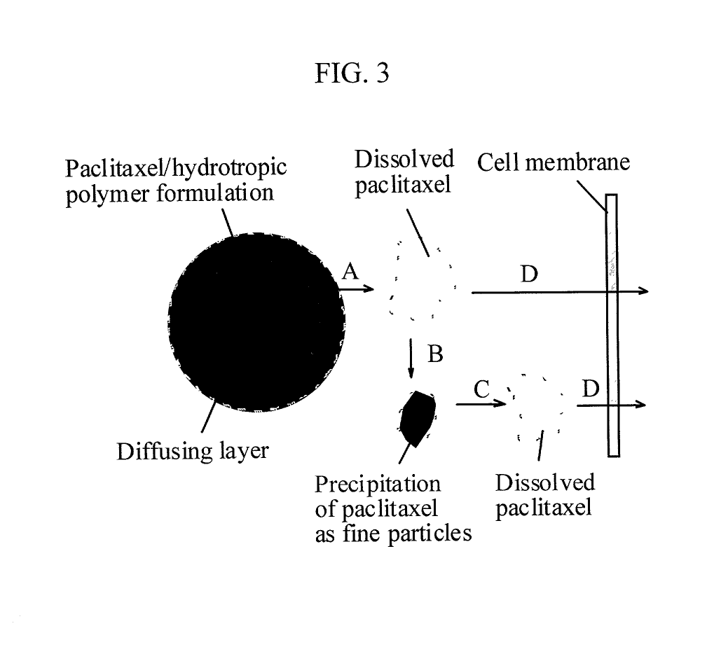

(3) Solid dispersions are prepared. Solid dispersion is a eutectic mixture of a poorly soluble drug and inert carrier that, upon exposure to aqueous solution, results in fine particles leading to faster dissolution and improved bioavailability. Although the solid dispersion method is an attractive approach for lipophilic drugs, only one drug, griseofulvin, is currently marketed in this form. The successful application of hydrotropic polymer solid dispersion of paclitaxel should reestablish the usefulness of this approach. Solid dispersions can be made by the fusion process, solvent method, or fusion-solvent method, depending on the melting temperatures and availability of suitable solvents for paclitaxel and hydrotropic polymers. Since the melting point of paclitaxel is 220° C., the fusion method is employed as long as the melting point of the hydrotropic polymers is lower than 200° C. The appropriate amount of hydrotropic polymer is weighed, placed in a porcelain crucible, and heated on a hotplate to melt. Paclitaxel is then added and melted with the hydrotropic polymers by mixing. The mixture is pipetted into open glass tubes with different diameters standing on a glass plate. Alternatively, the mixture can be spread on a clean glass plate to make thin films. After the dispersion is cooled to room temperature, the solid dispersion is carefully removed from the glass tube or glass plate. The solid dispersion is ground to make fine particles for easy administration. For in vitro paclitaxel release, the solid formulations are placed in a test tube with 1 ml water in a 37° C. water bath. At timed intervals, aliquots of the medium are taken out and filtered through a 0.22 μm nylon membrane for measurement of the paclitaxel concentration by HPLC. The release of paclitaxel from a solid dosage form and absorption through the cell membrane is illustrated in FIG. 3.

B. Cytotoxicity Evaluation of Hydrotropic Polymer Formulations.

Purdue Cancer Center Cell Culture Laboratory has provided bioassay service for measuring antitumor cytotoxicity for many years. Currently, the following human tumor cells are available for cytotoxicity evaluation: MCF-7 (breast), MCF-7ADR (breast, multidrug resistant), A-549 (lung), SK-OV-3 (ovary), PC-3 (prostate), and A-498 (kidney). The standard bioassay is done in 96-well microtiter plates using MTT [3-(4,5-dimethylthiazole-2-yl)-2,5-diphenytetrazolium bromide]. MTT is cleaved in the mitochondria of live cells to produce a dark blue formazan product. Thus, only live cells are stained and the staining intensity can be measured at 570 nm. Cytotoxicity is reported as GI₅₀, effective dose at which cell growth is retarded to 50% of the control culture. Adriamycin is used as an internal reference antitumor agent for the quality control of the standardized cytotoxicity assay.

The antitumor cytotoxicity, as measured by GI₅₀, of paclitaxel and adriamycin on various cell lines were measured as shown in Table 6. The results of cytotoxicity of paclitaxel in various hydrotropic excipient formulations (agent/polymer/gel) are examined and compared with the data in Table 6 to compare the effectiveness of the hydrotropic formulations. Both liquid and solid formulations are tested with varying concentrations (usually 5 different concentrations) of paclitaxel in the formulations. Free paclitaxel in Cremophor EL/ethanol (TAXOL) are used as a reference point for clinical effectiveness. The results of cytotoxicity evaluations are compared with those of animal experiments to examine what formulations were optimal for each experiment.

TABLE 6
GI₅₀ (μg/ml) of paclitaxel and adriamycin on various tumor cell lines¹

| Cancer cell lines | A-549 | MCF-7 | HT-29 | PC-3 | A-498 | PaCa-2 |
|---|---|---|---|---|---|---|
| Paclitaxel | 4 × 10⁻⁸ | 8 × 10⁻⁸ | 3 × 10⁻⁸ | 3 × 10⁻⁷ | 7 × 10⁻⁶ | 3 × 10⁻⁸ |
| Adriamycin | 5 × 10⁻³ | 2 × 10⁻¹ | 3 × 10⁻² | 2 × 10⁻² | 5 × 10⁻³ | 5 × 10⁻³ |

¹HT-29 (colon), PaCa-2 (pancreas)

C. P-Glycoproteins and the Paclitaxel Bioavailability

Successful oral delivery of paclitaxel requires overcoming of at least two hurdles: poor water-solubility, and pre-systemic elimination including intestinal and hepatic cytochromes P-450 metabolism and multi-drug resistant (MDR) transporters in the intestine. Expression of MDR transporters (that are also called phospho-glycoprotein (P-glycoprotein) or simply transporters) results in acquired resistance to anticancer agent. P-glycoproteins have evolved as protective systems to remove diverse substrates out of the cell, including toxic xenobiotics. Cell culture and in vivo studies in the literature have indicated that paclitaxel can be effectively absorbed from the intestinal tract, but its bioavailability is limited by P-glycoprotein. Oral bioavailability of paclitaxel in mice treated with a P-glycoprotein blocker was increased more than 10-fold. Currently available P-glycoprotein inhibitors are verapamil, cyclosporin A, Valspodar (a cyclosporine D analog), quinidine, quinine, quinoline derivative, tamoxifen, dexverapamil, cyclopropyldibenzosuberane, Cremophor EL, Solutol HS 15, ketoconazole, and vitamin E. It is not known whether the effect of P-glycoprotein on the absorption of paclitaxel from the GI tract is dependent on the concentration of paclitaxel, i.e., water-solubility of paclitaxel. P-glycoprotein may be a major deterrent of the absorption of paclitaxel when its concentration is low. As the concentration of paclitaxel increases, however, the absorption of paclitaxel should increase significantly due to the saturation of P-glycoprotein transporter efflux. Due to the lack of information on the concentration of P-glycoprotein in the GI tract, it is difficult to estimate the concentration of paclitaxel required to saturate P-glycoprotein. However, when the concentration of paclitaxel is increased to more than 1 mg/ml (more than 3 orders of magnitude increase in solubility), the effect of P-glycoprotein is expected to be overcome by abundant paclitaxel molecules. According to the one-compartment open model with first-order absorption and elimination, the amount of drug, A, in the body is described by the equation:

$$A = FD \frac{{k}_{a}}{{k}_{a} - {k}_{el}} ( {e}^{- {k}_{el} t} - {e}^{- {k}_{a} t} )$$

where F is the absorption efficiency, or the fraction of the dose, D, that is absorbed into the systemic circulation, Kₐ and Kₑₗ are absorption and elimination rate constants, and t is the time. The absorption efficiency, F, for paclitaxel may be very low due to the presence of P-glycoproteins in the GI tract. The point here is that as the dose, D, is increased, the total amount of paclitaxel absorbed is also increased. To be absorbed, the dose, D, has to be in solution. This is why the increase in water-solubility of paclitaxel is so important for increasing its oral bioavailability.

Adding polymeric excipients, such as alginate, gellan, and xanthan, to anticancer drugs minimizes the effect of P-glycoprotein on in vitro cell culture system and on in vivo oral absorption. Other polymers, such as PLURONIC, are also known to sensitize cancer cells to make them more vulnerable to the cancer drugs. If any of the hydrotropic polymers have P-glycoprotein inhibitory effect or sensitize cancer cells, it may increase the paclitaxel bioavailability even more. The effect of increased water solubility is not distinguished here from the effect of P-glycoprotein inhibition. The possible effect of hydrotropic polymers on transporters, such as P-glycoprotein, is of further interest.

D. Chronically Catheterized, Non-stressed Rat Model

A unique rat model utilizing techniques for chronic catheterization of major blood vessels and the intestinal tract has been developed and validated by Dr. Robert E. Kimura. Dr. Kimura taught the model to Dr. Galinsky while both were colleagues at the University of Utah and they have collaborated on several previous studies. This model, the subject of a laudatory commentary by Jared Diamond, has provided new insights into hepatic and intestinal physiology. The techniques used to catheterize the aorta, portal vein, inferior vena cava and stomach have been extensively described in several publications. In addition, bladder catheters for renal clearance studies and chronic gastric catheters for feeding liquid diets under normal physiologic conditions have been developed. Dr. Galinsky has successfully adapted this model to study the effects of parenteral nutrition on hepatic oxidative and conjugative metabolism. This model is unique and highly appropriate because the proposed studies are carried out in chronically catheterized animals that have returned to physiologic, non-stressed baseline conditions after surgery.

Rats have chronic catheters implanted in the inferior vena cava (for i.v. drug administration), in the duodenum (for oral drug administration), and in the aorta (for blood sampling). All rats have all three catheters to control for any surgery effects and to be able to use the rats as their own controls. On one occasion the animals receive drug through the i.v. catheter and on another occasion they receive drug through the duodenal catheter. Bioavailability can be computed by comparing the ratio of the AUC corrected for respective doses.

The paclitaxel formulation is administered to freely moving animals that have recovered not only from the surgery and anesthesia but also have regained preoperative weight, which usually occurs 3-4 days after surgery. Animals are not studied in the first few days after surgery, thereby avoiding artifacts due to bowel manipulation and anesthesia. Paclitaxel formulations are delivered through the duodenal catheter to avoid the potential that stomach emptying may become the rate-limiting step in absorption. In addition, this method allows delivery of larger volume (greater than 1.5 ml) to the duodenum whereas 1.5 ml is sometimes the largest amount that can be delivered to the stomach without the drug formulation coming back up the esophagus during administration. If delivery to the stomach is necessary, as a control study or to mimic the true oral delivery, the paclitaxel formulation is administered by gavages using an oral feeding needle (volume<1.5 ml).

Six rats per formulation and five doses (5-50 mg/kg) per formulation are used to define the concentration-dependence of paclitaxel bioavailability and clearance (if any). For each formulation, therefore, 30 rats are used. The use of rats is minimized by administering i.v. and oral paclitaxel to the same animals on two different occasions.

E. Pharmacokinetics Study of Paclitaxel

The bioavailability of paclitaxel is determined on rats at least 7 days or more after cannula implantation. Rats receive a single dose of paclitaxel ranging from 5-50 mg/kg, infused over 30 min via inferior vena cava catheter. Ten blood samples (250 μL each) are obtained via the aortic catheter over 12 hours after the start of the infusion. In some rats, portal vein catheters are implanted and blood samples are also obtained from the portal venous cannula at 1, 2, 4, 8, and 12 hours after the end of the infusion. This sampling schedule permits an accurate description of the AUC after i.v. or oral dosing. Following the pharmacokinetic study described above, the volume of blood removed by sampling (2.5 ml) is replaced with blood from a donor animal, which was not used for the bioavailability study. Pharmacokinetic analysis is performed using standard techniques. This study design permits calculation of hepatic clearance and availability to be determined for the various formulations to be tested. Except where specifically noted, the foundation for the pharmacokinetic analysis can be found in standard pharmacokinetics textbooks, such as Gibaldi and Perrier. The area under the curve (AUC) for paclitaxel in aortic blood is determined up to the last data point by a combination of linear and log-linear trapezoidal rules. The extrapolated area to infinity is determined from the quotient of the last measured serum concentration and the terminal elimination rate constant. That value is obtained from the terminal log-linear portion of the serum concentration time curves using log-linear regression. The systemic clearance (CL) of paclitaxel based on blood is determined from the intravenous (i.v.) dose (Doseᵢᵥ) and the serum AUC to infinity (AUC) for the i.v. dose using the equation:

CL=Doseᵢᵥ/AUCᵢᵥ.

It is also assumed that the “well-stirred” model functionally describes the dependence of hepatic clearance (CL_{H}) upon hepatic blood flow (Q_{H}), hepatic intrinsic clearance (CL_{INT,H}), and the fraction of paclitaxel unbound in blood (fᵤ) as shown in the equation:

CL_{B}=CL_{H}=(Q_{H}·fᵤCL_{INT,H}/(Q_{H}+fᵤ·CL_{INT,H}).

Fundamentally, this model assumes that the unbound concentration of drug at the hepatocyte metabolizing enzyme is equal to the unbound concentration leaving the liver. The well-stirred model has been used successfully to predict the in vivo clearance of midazolam from in vitro data.

The above equation allows estimation of CL_{INT,H} from the measured values of CL_{H} and fᵤ together with an estimated value of Q_{H}. The hepatic extraction ratio (E_{H}) and the hepatic availability (F_{H}) is calculated using the following equations:

E_{H}=CL_{H}/Q_{H}and F_{H}=1−E_{H}

The bioavailability (F) of paclitaxel is determined from:

F=(AUCₚₒDOSEᵢᵥ)/(AUCᵢᵥDOSE_{PO})

where AUC_{PO} is the area under the serum concentration versus time curve to infinity for oral dosing and DOSE_{PO} is the oral dose. For completeness, other pharmacokinetic parameters such as half-life (ln2/k), volume of distribution at steady state, mean residence time and mean absorption time are calculated for paclitaxel in the animals being studied for each of the formulations.

F. Determination of Paclitaxel Concentrations in Blood Samples

The concentrations of paclitaxel in the blood samples are determined by high performance liquid chromatography coupled to tandem mass spectrometry (HPLC-MS/MS). The blood samples are centrifuged at 3000 g for 10 min, and the plasma is transferred to 1.5 ml polypropylene tubes and kept at −70° C. until analysis. Frozen plasma samples are thawed at 37° C. in a water bath, and then paclitaxel is extracted with dichloromethane. These extracts are subjected to HPLC-MS/MS analysis. Desorption chemical ionization (DCI) MS/MS method is used to quantify paclitaxel in the HPLC effluent. Paclitaxel shows both an (M+H)⁺ and an (M+NH₄)⁺ ion under ammonia positive ionization conditions (M is the mass of paclitaxel). The compound becomes fragmented in a structurally characteristic fashion, and the MS/MS spectrum of the (M+H)⁺ ion is also structurally diagnostic. When 10 μg of plasma was examined by desorption chemical ionization, it gave the featureless mass spectrum. By contrast, the same amount of sample gave the product ion MS/MS spectrum. This allows ready identification of paclitaxel in the plasma.

Analysis of each plasma extract requires two measurements. First, 1 μl of the eluate is placed on the filament and the ion current for paclitaxel is recorded. Second, 1 μl of the sample is spiked with paclitaxel and reexamined. The spike is typically 1, 5, or 10 ng depending on the ion current recorded from the sample alone. This entire process takes approximately 10 min. The concentration of paclitaxel in the sample is determined from a standard curve of the ion abundance versus the amount of paclitaxel added. The limit of quantification of the paclitaxel in the plasma is less than 500 pg/ml.

V. Other Applications

A. Generation of a Sink Condition for Poorly Soluble Drugs

When a formulation of poorly soluble drug is prepared it is desirable to examine the drug release profile. To accurately measure the release kinetics, the release experiments should be done in a sink condition, i.e., a condition where the accumulated drug concentration in solution (C) is considerably less than the drug's solubility (C_{S}). Usually the sink condition is assumed if C is less than 10% of C_{S}. For paclitaxel, for example, C_{S} is 0.3 μg/ml, and thus, to maintain the sink condition, the paclitaxel concentration in solution should be less than 0.03 μg/ml. Thus, providing a sink condition for poorly soluble drugs requires a huge volume of aqueous medium compared with the volume of a sample. Furthermore, this leads to difficulty in measuring the exact amount of the released paclitaxel. In most cases, a large volume of aqueous medium is collected, freeze-dried, and the remaining drug is redissolved in organic solvent for analysis. This is not practical when dealing with numerous samples.

The use of hydrotropic agents, hytrops, and hytrogels eliminates this problem. Due to the very high solubility of poorly soluble drugs in hydrotropic agents, hytrops, and hytrogels, only a very small volume can be used as a release medium. This also allows analysis of the released drug as collected without going through a process of concentrating the drug.

B. Preparation of Aqueous Solutions of Poorly Soluble Drugs for in vitro Experiments and in vivo Animal Experiments.

The poor water solubilities of many drugs and drug candidates make it difficult to do experiments for identifying bioefficacy and dose-response studies. In most cases, poorly soluble drugs are dissolved in organic solvents and diluted in aqueous solution before the experiments. The use of hytrops and hytrogels can eliminate the problems associated with using organic solvents. Since the concentration of poorly soluble drugs can be very high in hytrops and hytrogels, very small amounts of aqueous solution can be used. A very small volume of hytrop and hytrogel formulations can be easily administered in animal experiments.

C. Preparation of Nano- and Micro-Particles of Poorly Soluble Drugs

As described hereinabove, the solubility of poorly soluble drugs can be increased by reducing the size of particles to micro- and nano-scales. The hydrotropic agents and hytrops are useful in making nano- and micro-particles of poorly soluble drugs. For example, paclitaxel is dissolved in an aqueous solution of N,N-diethylnicotinamide or its polymer. The solution is then sprayed as a nano- or micro-droplets using microdispensors into an aqueous solution containing surfactants. The hydrotropic agent or hytrop is diluted rapidly in abundant water due to their high water solubility, resulting in precipitation of paclitaxel particles. The size of the obtained particles depends on the size of the droplets, concentration and type of hydrotropic agent, and type of surfactants used. This is an easy way of preparing nano- or micro-particles of poorly soluble drugs. The following example highlights this particular application.

### EXAMPLE V-1

### Use of Hydrotropic Agent to Form Microparticles.

Paclitaxel is dissolved in N,N-diethylnicotinamide solution to make a final concentration of 5 (w/v) %. Microdroplets of the paclitaxel solution having a size of approximately 40 μm diameter are introduced into 10 ml of water using a microdispensor controlled by a single jet device. The water contains 0.1% Tween 21 to prevent aggregation of formed particles and the water is stirred using a magnetic stirring bar. The size distribution of the formed paclitaxel particles is measured by a microscope. The size ranges from 0.56 μm to 3.66 μm. The fractions of microparticles observed in the size ranges of less than 1 μm, 1-2 μm, 2-3 μm, and larger than 3 μm are 34.8%, 58.0%, 6.5%, and 0.7%, respectively. The majority of the formed paclitaxel microparticles is less than about 2 μm. Considering that the initial droplet size of the paclitaxel in N,N-diethylnicotinamide solution is 40 μm, it is expected that the paclitaxel particle size can be reduced even further to the nanometer range quite easily using microdispensers of smaller sizes. The advantages of this approach include its simplicity, avoidance of organic solvents, no need for expensive equipment and devices, and easy scale-up.

The present invention has been described hereinabove with reference to particular examples for purposes of clarity and understanding rather than by way of limitation. It should be appreciated that certain improvements and modifications can be practiced within the scope of the appended claims.

### REFERENCES

The pertinent portions of the following references are incorporated herein by reference:

Alkan-Onyuksel, H., S. Ramakrishnan, H. B. Chai and J. M. Pezzuto (1994). “A mixed micellar formulation suitable for the parenteral administration of taxol.” Pharm. Res. 11(2): 206-212.

Chiou, W. L. and S. Riegelman (1971). “Pharmaceutical applications of solid dispersion systems.” J. Pharm. Sci. 60: 1281-1302.

Coffman, R. E. and D. O. Kildsig (1996). “Effect of nicotinamide and urea on the solubility of riboflavin in various solvents.” J. Pharm. Sci. 85(9): 951-954.

Dhara, D. and P. R. Chatterji (1999). “Effect of hydrotropes on the volume phase transition in poly(N-isopropylacrylamide) hydrogel.” Langmuir 15: 930-935.

Floyd, A. G. and S. Jain (1998). Injectable emulsions and suspensions. Pharmaceutical Dosage Forms: Disperse Systems. H. A. Lieberman, M. M. Rieger and G. S. Banker. New York, N.Y., Marcel Dekker, inc. Vol. 2: 261-318.

Ford, J. L. (1986). “The current status of solid dispersions.” Pharm. Acta Helv. 61: 69-88.

Graziano, G. (2000). “On the solvent isotope effect in hydrophobic hydration.” J. Phys. Chem. B 104: 9249-9254.

Habib, M. J., S. Venkataram and M. D. Hussain (2001). Fundamentals of solid dispersions. Pharmaceutical Solid Dispersion Technology. M. J. Habib. Lancaster, Pa., Technomic Publishing Co., Inc.: 7-35.

Kumar, M. D. and N. N. Gandhi (2000). “Effect of hydrotropes on solubility and mass transfer coefficient of methyl salicylate.” J. Chem. Eng. Data 45: 419-423.

Leung, S. Y., J. Jackson, H. Miyake, H. Burt and M. E. Gleave (2000). “Polymeric micellar paclitaxel phosphorylates Bcl-2 and induces apoptotic regression of androgen-independent LNCaP prostate tumors.” Prostate 44(2): 156-163.

Löbenberg, R., G. L. Amidon and M. Vierira (2000). Solubility as a limiting factor to drug absorption. Oral Drug Absorption. Prediction and Assessment. J. B. Dressman and H. Lennernäs. New York, N.Y., Marcel Dekker: 137-153.

Macheras, P., C. Reppas and J. B. Dressman (1995). Biopharmaceutics of Orally Administered Drugs. New York, Ellis Horwood.

Mani, S., L. lyer, L. Janisch, X. Wang, G. F. Fleming, R. L. Schilsky and M. J. Ratain (1998). “Phase I clinical and pharmacokinetic study of oral 9-aminocamptothecin (NSC-603071).” Cancer Chemother. Pharmacol. 42(1): 84-87.

Martin, A., P. Bustamante and A. H. C. Chun (1993). Physical Pharmacy. Philadelphia, Pa., Lea & Febiger.

Müller, B. W. and E. Albers. (1991). “Effect of hydrotropic substances on the complexation of sparingly soluble drugs with cyclodextrin derivatives and the influence of cyclodextrin complexation on the pharmacokinetics of the drugs.” J Pharm. Sci. 80(6): 599-604.

Müller, R. H. and B. Bohm (1998). Nanosuspensions. Emulsions and Nanosuspensions for the Formulation of Poorly Soluble Drugs. R. H. Müller, S. Benita and B. Böhm. Stuttgart, Germany, Medpharm Scientific Publ.: 149-174.

Nicolaou, K. C., C. Riemer, M. A. Kerr, D. Rideout and W. Wrasidlo (1993). “Design, synthesis and biological activity of protaxols.” Nature 364(6436): 464-466.

Pendri, A., C. D. Conover and R. B. Greenwald (1998). “Antitumor activity of paclitaxel-2′-glycinate conjugated to poly(ethylene glycol): a water-soluble prodrug.” Anticancer Drug Des. 13(5): 387-395.

Saleh, A. M., A. R. Ebian and M. A. Etman (1986). “Solubilization of water by hydrotropic salts.” J. Pharm. Sci. 75(7): 644-647.

Serajuddin, A. T. M. (1999). “Solid dispersion of poorly water-soluble drugs,” J. Pharm. Sci. 88: 1058-1066.

Sharma, A. S. R. M. (1994). “Novel taxol formulations: preparation and characterization of taxol-containing liposomes.” Pharm. Res. 11(Jun): 889-896.

Srinivas, V. and D. Balasubramanian (1998). “When does the switch from hydrotropy to micellar behavior occur?” Langmuir 14: 6658-6661.

Tavare, N. S. and V. K. Jadhav (1996). “Solubilities of 6-aminopenicillanic acid and phenoxyacetic acid in hydrotrope solutions.” J. Chem. Eng. Data 41(5): 1196-1202.

Yalkowsky, S. H. (1999). Solubility and Solubilization in Aqueous Media. Washington, DC, American Chemical Society, p. 429.

What is claimed is:

1. A pharmaceutical composition comprising a pharmacologically effective amount of a poorly soluble drug and a solubilizing compound selected from the group consisting of hydrotropic agent monomers, hydrotropic polymers, and hydrotropic hydrogels, wherein the solubilizing compound includes at least one hydrophobic moiety.

2. The composition of claim 1; wherein the solubilizing compound is a hydrotropic polymer or hydrotropic hydrogel.

3. The composition of claim 1, wherein the hydrophobic moiety is selected from the group consisting of substituted and unsubstituted aryl groups, substituted and unsubstituted nitrogen heterocycles, alkyl groups, alkylene groups, aralkyl groups, and methacryloyl groups.

4. The composition of claim 1, wherein the hydrophobic moiety is a substituted or unsubstituted pyridyl group.

5. The composition of claim 1, wherein the hydrophobic moiety is selected from the group consisting of N,N-diethylnicotinamide, N-picolylnicotinamide, N-allynicotinamide, sodium salicylate, 2-methacryloyloxyethyl phosphorylcholine, resorcinol, N,N-dimethylnicotinamide, N-methylnicotinamide, butylurea, pyrogallol, 3-picolylacetamide, procaine HCl, nicotinamide, pyridine, 3-picolylamine, sodium ibuprofen, sodium xylenesulfonate, and ethyl carbamate.

6. The composition of claim 1, wherein the poorly soluble drug has a solubility in water of less than about 100 μg/ml at 37° C.

7. The composition of claim 1, wherein the poorly soluble drug is selected from the group consisting of paclitaxel, griseofulvin, progesterone, and tamoxifen.

8. A hydrotropic polymer or copolymer capable of increasing water solubility of a poorly soluble drug, wherein the polymer or copolymer comprises at least one hydrotropic agent monomer unit that includes a hydrophobic moiety.

9. The polymer or copolymer of claim 8, wherein the hydrophobic moiety is selected from the group consisting of substituted and unsubstituted aryl groups, substituted and unsubstituted nitrogen heterocycles, alkyl groups, alkylene groups, aralkyl groups, and methacryloyl groups.

10. The polymer or copolymer of claim 8, which has a block, graft, alternating or random arrangement of monomer units.

11. The polymer or copolymer of claim 8, which has an acrylate or methacrylate backbone.

12. The polymer or copolymer of claim 8, which contains a spacer group.

13. The polymer of claim 8, which is a homopolymer of a hydrotropic agent monomer.

14. The polymer or copolymer of claim 8, wherein the hydrotropic agent monomer unit is selected from the group consisting of polymerizable derivatives of nicotinamide, N-substituted nicotinamide, pyridinium, N-substituted pyridinium, benzyl, urea, thiourea, pyridone, pyrimidone, melamine, pyridine, pyrazine, nicotine, triazine, salicylamide, salicylic acid, and sulfimide.

15. The polymer or copolymer of claim 8, wherein the at least one hydrotropic agent monomer unit is selected from the group consisting of vinyl derivatives of ibuprofen, nicotinamide, salicylic acid, N-picolylnicotinamide, salicylaldehyde, N,N′-dimethylnicotinamide, N,N′-diethylnicotinamide, and pyridine.

16. The polymer or copolymer of claim 8, wherein the poorly soluble drug has a solubility in water of less than about 100 μg/ml at 37° C.

17. The polymer or copolymer of claim 16, wherein the poorly soluble drug is paclitaxel, griseofulvin, progesterone, or tamoxifen.

18. A hydrotropic hydrogel capable of increasing water solubility of a poorly soluble drug, wherein the hydrogel is formed by polymerizing at least one hydrotropic agent monomer in the presence of a crosslinking agent.

19. The hydrogel of claim 18, wherein the poorly soluble drug has a solubility in water of less than about 100 μg/ml at 37° C.

20. The hydrogel of claim 18, which is capable of increasing the solubility of paclitaxel.

21. A method of increasing water solubility of a hydrophobic compound comprising combining the hydrophobic compound with a solubilizing compound selected from the group consisting of hydrotropic agents, hydrotropic agent monomers, hydrotropic polymers, and hydrotropic hydrogels, wherein the solubilizing compound includes a hydrophobic moiety.

22. A method of administering a poorly soluble drug to a patient comprising administering to the patient a composition containing the drug and a solubilizing compound selected from the group consisting of hydrotropic agents, hydrotropic agent monomers, hydrotropic polymers, and hydrotropic hydrogels, wherein the solubilizing compound includes a hydrophobic moiety.

23. The method of claim 22, wherein the composition is administered orally.

24. The method of claim 22, wherein the poorly soluble drug has an aqueous solubility of less than about 100 μg/ml at 37° C. in the absence of said solubilizing compound.

25. A method of making a hydrotropic polymer comprising polymerizing at least one hydrotropic agent monomer, wherein the monomer contains a hydrophobic moiety.

26. A method of making a hydrotropic polymer comprising grafting a hydrotropic agent to a polyacrylate, polymethacrylate, polyacrylamide, polyol, or polyamine backbone.

27. A method of making a hydrotropic hydrogel comprising polymerizing at least one hydrotropic agent monomer in the presence of a crosslinking agent.

28. A method of forming a solid dispersion of a poorly water-soluble drug and a solubilizing agent selected from the group consisting of hydrotropic agents, hydrotropic agent monomers, hydrotropic polymers, and hydrotropic hydrogels, said solubilizing compound containing a hydrophobic moiety, comprising:
melting the drug in the presence of the solubilizing compound; and
allowing the resulting composition to cool.

29. A method of removing lipids from a lipid-containing extract comprising contacting the extract with a solubilizing compound selected from the group consisting of hydrotropic agents, hydrotropic agent monomers, hydrotropic polymers and hydrotropic hydrogels.

30. A method of forming submicron sized particles of a poorly soluble drug comprising:
forming an admixture of the drug and a solubilizing compound selected from the group consisting of hydrotropic agents, hydrotropic agent monomers, hydrotropic polymers and hydrotropic hydrogels; and
diluting the admixture in water, thereby precipitating the drug particles.
